(19) United States
(12) Patent Application Publication
(10) Pub. No.: US 2022/0047719 A1
(43) Pub. Date: Feb. 17, 2022

(54) ENHANCING SUBCUTANEOUS INJECTION AND TARGET TISSUE ACCUMULATION OF NANOPARTICLES VIA CO-ADMINISTRATION WITH MACROPINOCYTOSIS INHIBITORY NANOPARTICLES (MINP)

(71) Applicant: Northwestern University, Evanston, IL (US)

(72) Inventors: Evan Alexander Scott, Evanston, IL (US); Trevor Stack, Evanston, IL (US)

(21) Appl. No.: 17/445,285

(22) Filed: Aug. 17, 2021

Related U.S. Application Data
(60) Provisional application No. 63/066,596, filed on Aug. 17, 2020.

(51) Int. Cl. A61K 47/69; A61K 45/06

(52) U.S. Cl. CPC A61K 47/6935; B82Y 5/00; A61K 47/6907; A61K 45/06

(57) ABSTRACT

Provided herein are multi-nanoparticle formulations. Also provided herein are methods for enhancing circulation time and/or cell-targeting efficacy of an effector nanoparticle by administering an endocytosis inhibitory nanoparticle and an effector nanoparticle.

### CROSS-REFERENCE TO RELATED APPLICATIONS

This application claims the benefit of priority under 35 U.S.C. § 119(e) to U.S. Provisional Application No. 63/066,596 filed Aug. 17, 2020, the contents of which are incorporated herein by reference in their entireties.

### STATEMENT REGARDING FEDERALLY SPONSORED RESEARCH

This invention was made with government support under 1DP2HL132390-01 awarded by National Institutes of Health, 1806007 awarded by the National Science Foundation, and 1453576 awarded by the National Science Foundation. The government has certain rights in the invention.

### BACKGROUND OF THE INVENTION

Nanoparticles are versatile carriers that can improve and often specify the stability, circulation time, and biodistribution of therapeutic molecules. Despite these advantages, rapid clearance of nanoparticles by the mononuclear phagocyte system (MPS) remains a significant barrier to their applications in precision medicine. The MPS consists of circulating and organ-resident phagocytic cells, which internalize nanoparticles and eventually clear them through the liver. A comprehensive survey of the literature reported that a median average of only 0.7% of administered nanoparticles successfully reach solid tumors despite the use of surface-conjugated targeting moieties like antibodies, peptides or aptamers. Clearance by MPS cells occurs primarily in the liver, spleen and lymph nodes through a number of endocytic pathways including clathrin-mediated and clathrin-independent pathways, macropinocytosis, and phagocytosis. Macropinocytosis is a process by which membranes extend and form around extracellular fluid leading to internalization of the encapsulated region, while phagocytosis is usually receptor-initiated and internalizes with or without extension of plasma membranes through the use of membrane invaginations. If these pathways are temporarily inhibited in MPS cells prior to or in conjunction with the introduction of therapeutic nanoparticles, the bioavailability and therapeutic efficacy of these nanoparticles would likely increase significantly. Developing nanoparticles with “stealth” properties to avoid this non-specific uptake remains a critical objective for nanomedicine and many different strategies such as PEGylation and CD47 “don't eat me” peptides, have been tried with variable levels of effectiveness. Combinatorial strategies employing multiple different stealth strategies are needed to further reduce clearance by the MPS and increase nanomaterial utility in vivo.

There is a need for a nanoparticle formulation that prevents nonspecific clearance of nanoparticles by the MPS. In particular, there is a need for methods and formulations that addresses these cellular and biochemical mechanisms of nanoparticle clearance to improve therapeutic targeting of cells, such as within tumors.

### SUMMARY OF THE INVENTION

The present disclosure provides multi-nanoparticle formulations. In one aspect, the multi-nanoparticle formulation comprises (1) an endocytosis inhibitory nanoparticle (for example, a macropinocytosis inhibitory nanoparticle (MiNP)) and (2) an effector nanoparticle (E-NP). In some embodiments, the endocytosis inhibitory nanoparticle comprises (a) a nanostructure comprising a poly(ethylene glycol)-block-poly(propylene sulfide) (PEG-b-PPS) copolymer and (b) an endocytosis inhibitor loaded in the nanostructure. In some embodiments, the nanostructure is a micelle, in other embodiments, the nanostructure is a bicontinuous nanosphere. In some embodiments, the endocytosis inhibitory nanoparticle is a micropinocytosis inhibitory nanoparticle (MiNP) and the endocytosis inhibitor is a micropinocytosis inhibitor.

The present disclosure also provides methods for enhancing circulation time and/or cell-targeting efficacy of an effector nanoparticle in a subject. In one aspect, the method comprises the steps of (1) administering an endocytosis inhibitory nanoparticle (for example, a macropinocytosis inhibitory nanoparticle (MiNP)) and (2) administering an effector nanoparticle. In some embodiments, the endocytosis inhibitory nanoparticle comprises (a) a nanostructure comprising a poly(ethylene glycol)-block-poly(propylene sulfide) (PEG-b-PPS) copolymer and (b) an endocytosis inhibitor loaded in the nanostructure. In some embodiments, the nanostructure is a micelle. In other apsects, the nanostructure is a bicontinuous nanosphere. In some embodiments, the endocytosis inhibitory nanoparticle is a micropinocytosis inhibitory nanoparticle (MiNP) and the endocytosis inhibitor is a micropinocytosis inhibitor.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Schematic of tumor bearing mice being co-administered with latrunculin A-loaded macropinocytosis inhibitory nanoparticles (MiNP). MiNP were developed and evaluated for their effect on the accumulation of a targeted “effector” nanoparticle via subcutaneous and intravenous injection. As MiNP interferes with macropinocytosis but not receptor-mediated endocytosis, pre- and/or co-injection of MiNP with an effector nanoparticle displaying targeting ligands allows enhanced uptake by cells expressing the target receptor. As an example, MiNP are shown enhancing the targeting of receptors highly expressed within tumor microenvironments by interfering with off-target mononuclear phagocyte system (MPS) clearance.

FIG. 2. LatA retains its endocytic inhibition properties and does not change the size of PEG-b-PPS micelles when encapsulated. (a) Cryogenic transmission electron microscopy (CryoTEM) of MiNP visually confirms retention of micellar structures. (b) MiNP with ((+) MiNP) and without ((−) MiNP) loaded LatA were characterized via small angle X-ray scattering (SAXS) and fitted with a micelle model fit using SASView. (c) Free LatA and (+) MiNP significantly inhibited macropinocytosis by RAW264.7 macrophages as compared to clathrin-mediated endocytosis inhibitor chlorpromazine. Cells were treated with each inhibitor for 2 h followed by 30 min of incubation with pHrodo dextran prior to analysis by flow cytometry. Data are shown as a percentile scale of endocytosis inhibition. On this scale, 0% represents standard cell uptake with no inhibitor, while 100% represents complete inhibition with no uptake of dye. N=3, p<0.001. (d) In comparison, uptake of transferrin conjugated pHrodo dextran by macrophages via receptor-mediated endocytosis (RME) was significantly inhibited by chlorpromazine compared to (+) MiNP. N=3, p<0.001. (e) Loading within (+) MiNP significantly decreased the toxicity of LatA. Macrophages were incubated with various doses of free LatA or (+) MiNP for 4 h and assessed by flow cytometry for viability via the Zombie Aqua live/dead assay. N=3, p<0.05.

FIG. 3. Latrunculin A loaded MC ((+) MiNP) co-injection and −4 h pre-injection lead to similar effector particle biodistributions. (a) Timeline showing the injection times for the co-injection and −4 h injection methods, which were evaluated for both subcutaneous (SC) and intravenous (IV) administration. “(±) MiNP” indicated an injection of either (+) MiNP or (−) MiNP, and effector micelle injections are indicated by “E-MC”. All mice were sacrificed at 24 h post E-MC injection. Comparisons of cell uptake in spleen and liver for the different SC (b and c) and IV (d and e) injection methods are shown. In all cases, mice were injected with 100 μL 7 μM LatA (+) MiNP or (−) MiNP and E-MC were labelled with DiR for flow cytometric quantification of cellular uptake within the spleen and liver. Data are reported as fold increase median fluorescence intensity of the E-MC over an untreated control. N=5, p<0.0001. To assess the transience of the MiNP effect, mice were injected SC (f) or IV (g) with (±) MiNP and E-MC according to the co-injection method, and serum levels of E-MC were evaluated by fluorescence spectroscopy. Mice were then rested for 72 hours and injected again with only E-MC to determine whether the inhibitory effect remained. N=3 for 2 h and 4 h timepoints and N=6 for 24 h timepoints, *p<0.05.

FIG. 4. (+) MiNP treatment increases the accumulation of folate-targeted E-MC (E-MC(FA)) in B16F10 tumors following SC injection. (a) Timeline of injection protocol assessing the tumor-targeting co-injection method. (±) MiNP indicates an injection of either (+) MiNP or (−) MiNP. Mice were sacrificed 24 h after the co-injection for analysis by flow cytometry. Results are shown for IV (b-d) and SC (e-g) injections of 3 co-injection modalities: (−) MiNP treatment/E-MC, (+) MiNP treatment/E-MC, and (+) MiNP/E-MC(FA). Fluorescent E-MC and E-MC(FA) uptake by 3 different cell subsets were quantified: non-immune cells (b and e), dendritic cells (c and f), and macrophages (d and g) for 4 different organs. Data are reported as fold increase median fluorescence intensity of E-MC or E-MC(FA) over a PBS baseline control. N=4-10, p<0.05. Significance was determined within each organ by separate unpaired student's t-tests.

FIG. 5. Stability data for encapsulated molecules over the course of 7 days for E-MC, (+) MiNP, and E-MC(FA). Particles were dialyzed in water and samples were taken at day 0, 1, 3, and 7. Percentage of the initially encapsulated or incorporated molecule is reported for n=3 technical replicates. E-MC DiI (A), (+) MiNP DiR(B), (+) MiNP Lat A (C), E-MC(FA) DiI (D), and E-MC(FA) Folic Acid (E) content are reported.

FIG. 6. Flow cytometry gating strategy for dendritic cells (DCs) and macrophages. Cells were obtained from the spleen of mice injected with DiR labelled nanomaterials. Immune cells described in the manuscript are determined as follow: DCs: CD45⁺, CD11c⁺, CD19⁻. Macrophages: CD45⁺, F4/80⁺, CD19⁻.

FIG. 7. Custom lipid amphiphiles allow PEG-b-PPS micelles to incorporate and display folate with high efficiency. Folate-PEG-Lipid constructs were evaluated for their ability to partition into PEG-b-PPS micelles using spectroscopy by assessing the intrinsic absorption of folate. The amount of folate loaded onto micelles is shown in comparison to the amount of folate added for 3 different molar ratios (a-c) of PEG-b-PPS polymer to folate-PEG-lipid construct.

FIG. 8. Folate targeted ‘effector’ micelles (E-MC(FA)) are taken up at a higher rate by B16F10 cells than non-targeted E-MC. B16F10 cells were incubated with non-targeted E-MC or folate receptor targeted E-MC(FA) for 1 hour and uptake was quantified by flow cytometry. Data are reported as median fluorescence intensity of E-MC or E-MC(FA). Statistical significance determined by unpaired student's t-test. N=3, p<0.005.

FIG. 9. Serum content of E-MC in tumor bearing mice after administration of either (+) MiNP or (−) MiNP 24 h post E-MC injection. Data are expressed as fold increase of E-MC fluorescence over the baseline, which was defined by the average of 3 mice injected with nanoparticles lacking fluorescence. Significance was determined by unpaired student's t-test. N=4-5, p<0.05.

FIG. 10. Latrunculin A inhibits uptake of non-targeted E-MC more than targeted E-MC(FA). B16F10 melanoma cells were treated with 0.5 μM LatA for 2 h followed by 30 min of incubation with either non-targeted E-MC or folate receptor targeted E-MC(FA) prior to analysis by flow cytometry. Data are shown as percentage inhibition of endocytosis normalized to untreated macrophages. N=3, p<0.001.

FIG. 11. BCN lysosomal persistence and loading capacity. (A) Percentage of nanoparticle positive (NP+) cells at 8 h and 36 h determined in Schlemm's canal endothelial cells using high throughput fluorescence microscopy. Significant changes in % NP+ cells at 8 h and 36 h were determined using a two-tailed t-test and a 5% significance level. ****p<0.0001; *p<0.05. (B) LatA loading capacity comparison in PEG-b-PPS micelles versus BCNs. Significant differences in LatA loading was determined using a two-tailed t-test and a 5% significance level. *p<0.05.

FIG. 12. LatA loading does not disrupt the internal cubic organization of the BCN or FM morphology. LatA-loaded and blank (no drug) PEG-b-PPS (A) BCN (B) and FM were analyzed using SAXS. Bragg peaks with the √V2, √V4, and √V6 spacing ratios indicate the presence of primitive type (1 m3 m) internal cubic organization was retained for BCN. Flexible cylinder models were fit to the FM data (dashed lines). Modeling details are as follows: LatA FM: 1.52 um length, 380 nm Kuhn length, 191 nm persistence length, 16.2 nm radius, X²=0.23. Blank FM: 1.51 um length, 380 nm Kuhn length, 190 nm persistence length, 16.2 nm radius, X²=0.36.

### DETAILED DESCRIPTION OF THE INVENTION

The present invention has been described in terms of one or more preferred embodiments, and it should be appreciated that many equivalents, alternatives, variations, and modifications, aside from those expressly stated, are possible and within the scope of the invention.

The present disclosure provides multi-nanoparticle formulations that can be utilized to enhance circulation time and/or cell-targeting efficacy of an effector nanoparticle. In particular embodiments, administration of the multi-nanoparticle formulations allows for inhibiting the nonspecific clearance of nanoparticles by endocytosis, including the mononuclear phagocytic system (MPS). In some embodiments, the multi-nanoparticle formulations can also be utilized in cancer nanotherapy by increasing the accumulation of the nanoparticles within the tumor microenvironment.

In one embodiment, the disclosure provides multi-nanoparticle formulations. In one embodiment, the multi-nanoparticle formulation comprises (1) an endocytosis inhibitory nanoparticle, for example, a macropinocytosis inhibitory nanoparticle (MiNP) and (2) an effector nanoparticle (E-NP). In one embodiment, the an endocytosis inhibitory nanoparticle comprises (a) a nanostructure comprising a poly(ethylene glycol)-block-poly(propylene sulfide) (PEG-b-PPS) copolymer and (b) a endocytosis inhibitor, for example, a macropinocytosis inhibitor, loaded in the nanostructure. In one embodiment, the endocytosis inhibitory nanoparticle is a micropinocytosis inhibitory nanoparticle (MiNP). In some embodiments, the endocytosis inhibitory nanoparticle is a micropinocytosis inhibitory nanoparticle (MiNP) and the endocytosis inhibitor is a micropinocytosis inhibitor.

Endocytosis inhibitor nanoparticle refer to nanostructures or nanoarchitectures capable of encapsulating or comprising as part of the nanoparticle an endocytosis inhibitor that allows for inhibiting endocytic uptake of an effector nanoparticle. Macropinocytosis inhibitory nanoparticle (MiNP) described herein are characterized by complex or vesicular nanoarchitectures capable of encapsulating or comprising as part of the nanoparticle a macropinocytosis inhibitor that allows for inhibiting the uptake of an effector nanoparticle by the mononuclear phagocyte system (MPS) within a subject.

In some embodiments, the nanostructure is a micelle, e.g., solid-core spherical micelles (e.g., PEG₄₅-b-PPS₂₉, 20-40 nm diameter, MC). Specifically, the micelle is capable of inhibiting clearance of the effector nanoparticle (E-NP) in the multi-nanoparticle formulation by the MIPS via macropinocytosis before reaching the targeted cells. The MiNP may comprise other suitable nanostructures, for example, poly(ethylene glycol)-b-poly(propylene sulfide) (PEG-b-PPS) copolymers can be made into diverse nanostructures: solid-core spherical micelles (PEG₄₅-b-PPS₂₉, 20-40 nm diameter, MC), vesicular polymersomes (PEG₁₇-b-PPS₃₀, 100-150 nm diameter, PS), filamentous worm-like micelles (PEG₄₅-b-PPS₄₄, 10-30 nm diameter×1-2 micron length, FM) and bicontinuous nanospheres (PEG₁₇-b-PPS₇₅, 150-250 nm diameter, BCN). Each morphology has a unique organ and cellular biodistribution upon in vivo administration, which enables passive targeting strategies without the need for targeting ligands. Many of these structures are difficult to consistently form with other polymer systems, making PEG-b-PPS a uniquely versatile tool for nanoparticle fabrication. Furthermore, PEG-b-PPS forms lyotropic mesophases, and this liquid crystallinity plays a critical role in its ability to kinetically trap aggregate morphologies and enhances overall aggregate stability under a range of conditions. Additional advantages of the PEG-b-PPS nanoparticle system include rapid gram-scale fabrication, stability for months, high loading efficiency for protein and small molecules, redox-sensitivity for intracellular delivery and enhanced endosomal escape, morphology-dependent passive targeting of myeloid cells, amenability to multimodal imaging, and multi-payload delivery. Of note, PEG-b-PP S is nontoxic in mice and nonhuman primates at up to 200 mg/kg⁴⁵.

In some embodiments, the MiNP comprises (a) a nanostructure comprising a poly(ethylene glycol)-block-poly(propylene sulfide) (PEG-b-PPS) copolymer and (b) a macropinocytosis inhibitor loaded in the nanostructure. In one suitable embodiment, the nanostructure is a micelle or bicontinuous nanosphere. Bicontinuous nanospheres (BCN) comprise highly stable inverted colloids characterized by a cubic lattice of aqueous channels that traverse a hydrophobic interior volume. BCN are the polymeric equivalent of lipid cubosomes, and their organized, lyotropic and interconnected internal architecture makes them exceptionally robust and stable assemblies. Relative to other nanostructures, like spherical micelles and vesicular polymersomes or liposomes, BCN are particularly amenable to high capacity dual loading of both hydrophobic and hydrophilic cargo, which respectively partition into the hydrophobic volume and aqueous channels. Applications of BCN have been limited by difficulties with fabrication, which the inventors have addressed using self-assembling BCN from PEG-b-PPS copolymers using flash nanoprecipitation (FNP), which remains the only method for their scalable uniform fabrication verified by dynamic light scattering, cryogenic and transmission electron microscopy, and small-angle X-ray scattering (SAXS). SAXS is required to verify the cubic interior, which is marked by Bragg peaks with relative spacing ratios at √2, √4, and √6, indicating the presence of primitive type (Im3 m) cubic internal organization. PEG-b-PPS BCN can release payloads in response to physiological levels of reactive oxygen species. Of note, the internal aqueous channels allow BCN to slowly release payloads locally in a size-dependent manner, functioning as nanoscale analogs to more classical macroscale porous hydrogels. BCN is a platform for either triggered or sustained intracellular drug release.

In one embodiment, the PEG-b-PPS copolymer is a PEG₄₅-b-PPS₂₃ copolymer. Suitable preparation of PEG-b-PPS nanocarriers disclosed herein can be prepared via known methods, e.g., Du, F., et al., (2019): Homopolymer Self-Assembly via Poly(propylene Sulfone) Networks. ChemRxiv. Preprint; Du F. et al., Sequential intracellular release of water-soluble cargos from Shell-crosslinked polymersomes. J Control Release. 2018; 282: 90-100; and Yi S., et al, Tailoring Nanostructure Morphology for Enhanced Targeting of Dendritic Cells in Atherosclerosis. ACS Nano. 2016; 10(12): 11290-11303, each of which are incorporated herein by reference in their entirety.

The nanoparticle is formed from a poly(ethylene glycol)-block-poly(propylene sulfide) (PEG-b-PPS) copolymer, which is an amphiphilic copolymer. Amphiphilic copolymers are comprised of sub-units or monomers that have different hydrophilic and hydrophobic characteristics. Typically, these sub-units are present in groups of at least two, comprising a block of a given character, such as a hydrophobic or hydrophilic block. Depending on the method of synthesis, these blocks could be of all the same monomer or contain different monomer units dispersed throughout the block, but still yielding blocks of the copolymer with substantially hydrophilic and hydrophobic portions. These blocks can be arranged into a series of two blocks (diblock) or three blocks (triblock), or more, forming the backbone of a block copolymer. In addition, the polymer chain may have chemical moieties covalently attached or grafted to the backbone. Such polymers are graft polymers. Block units making up the copolymer can occur in regular intervals or they can occur randomly making a random copolymer. In addition, grafted side chains can occur at regular intervals along the polymer backbone or randomly making a randomly grafted copolymer. The ratio of the hydrophobic to hydrophilic blocks of the copolymer will be selected such that the soluble and insoluble components are balanced and suitable aggregation for the desired architectures.

The poly(ethylene glycol)-block-poly(propylene sulfide) (PEG-b-PPS) copolymer of the present invention may also include chemical modifications or end caps. Chemical modification and end caps may include, but are not limited to, thiol, benzyl, pyridyl disulfide, phthalimide, vinyl sulfone, aldehyde, acrylate, maleimide, and n-hydroxysuccinimide groups. The chemical modification of the copolymer may add a charged residue to the polymer or may be used to otherwise functionalize the polymer. The ability to functionalize the polymer allows for suitable reactivities for further modification, if required or wanted.

As used herein, the term “micelle” refers a nanocarrier having a PEG weight fraction above 0.38. One skilled in the art would be able to determine proper weight fractions and may vary from the examples provided herein but still be within the scope of the invention. In some embodiments, the micelle has a hydrophobic/lipophilic core and a hydrophilic exterior. Micelle nanocarriers have a spherical morphology and are typically smaller (e.g., less than 50 nm diameter) than polymersomes and the hydrophobic core can be loaded with a lipophilic payload molecule or therapeutic agent. The micelles suitably have a PEG weight fraction of about 0.38 to about 0.69. Micelles can be prepared via known methods, for example those described in Karabin, N. B., Allen, S., Kwon, H. et al. Sustained micellar delivery via inducible transitions in nanostructure morphology. Nat Commun 9, 624 (2018), which is incorporated herein by reference.

In some embodiments, the micelles may have a diameter of from 5 nm to 60 nm, alternatively from 8 nm to 57 nm, alternatively from 11 nm to 54 nm, alternatively from 14 nm to 51 nm, alternatively from 17 nm to 48 nm, alternatively from 20 nm to 45 nm, alternatively from 23 nm to 42 nm, alternatively from 26 nm to 39 nm, alternatively from 29 nm to 36 nm, alternatively from 31 nm to 33 nm.

In some embodiments of the present invention, the polymer of the nanoparticle is PEG₄₅-bl-PPS₂₃. Advantages of the PEG-b-PPS nanocarrier system include rapid gram-scale fabrication, stability, high loading efficiency for proteins (e.g., ˜70% for albumin) and small molecules (e.g, >90% for imiquimod derivatives), and redox-sensitivity for intracellular delivery, amenability to multimodal imaging. In some embodiments of the present invention, the nanoparticle has a loading efficiency of the therapeutic agent of from 0.01% to 100%, alternatively 5% to 100%, alternatively 5% to 95%, alternatively 50% to 100%, alternatively 50% to 98%, alternatively 50% to 95%.

In some embodiments, the nanoparticle has a loading efficiency of the therapeutic agent of from 0.1% to 98%, or from 1% to 95%, or from 5% to 90%, or from 10% to 85%, or from 15% to 80%, or from 20% to 75%, or from 25% to 70%, or from 30% to 65%, or from 35% to 60%, or from 40% to 55%, or from 45% to 50%. In some embodiments, the loading efficiency is from 56% to 66%, or from 57% to 64%, or from 59% to 63%. As used herein, the term “endocytosis inhibitor” refers to a molecule or drug capable of inhibiting endocytosis or non-specific uptake of effector nanoparticles from a cell. Suitable endocytosis inhibitors include micropinocytosis inhibitors. Suitable endocytosis inhibitors that can be used in the present invention are known by oen skilled in the art and include, for example, those listed in Dutta D, Donaldson J G. Search for inhibitors of endocytosis: Intended specificity and unintended consequences. Cell Logist. 2012; 2(4): 203-208. doi:10.4161/cl.23967 and incorporated by reference in its entirety (e.g., hypertonic sucrose, chlorpromazine, monodansylcadaverine, phenylarsine oxide, chloroquine, monensin, phenothiazines, methyl-β-cyclodextrin, filipin, cyrochalasin D, latrunculin, amiloride, dynasore, dynoles, dyngoes, pitstop 2, dynamin mutant (DynK44A), siRNA of clathrin, siRNA of AP2, etc.), or those listed in Georgeta Basturea, MATER METHODS 2019; 9:2752 (//dx.doi org/10.13070/mm.en.9.2752), such as found in Table 2, incorporated by reference in its entirety regarding inhibitors (e.g., chlorpromazine (C₁₇H₁₉CIN₂S—HCl), Genistein (C₁₅H₁₀O₅), β-cyclodextrin (C₄₂H₇₀O₃₅), Amiloride Hydrochloride (C₆H₈ClN₇O.HCl.H₂O), Dynasore (C₁₈H₁₄N₂O₄), Dyngo 4a (dynasore analog, C₁₈H₁₄N₂O₅), MiTMAB™ (C₁₇H₃₈BrN), OcTMAB™ (C₂₁H₄6BrN), Filipin (C₃₅H₅₈O₁₁), Nystatin (C₄₇H₇₅NO₁₇), Monensin (C₃₆H₆₁O₁₁Na), Chloroquine diphosphate (C₁₈H₂₆ClN₃.H₃PO₄), Wortmannin (C₂₃H₂₄O₈), Pitstop 2 (C₂₀H₁₃BrN₂O₃S₂), Casin (C₂₀H₂₂N₂O), among others. As used herein, the term “macropinocytosis inhibitor” refers to a molecule or drug that is capable of inhibiting macropinocytosis or non-specific uptake of the effector nanoparticles by the MPS. Suitable agents for inhibiting macropinocytosis are known in the art and include, for example, latrunculin A (LatA), latrunculin B (LatB), among others. In some embodiments, the macropinocytosis inhibitor is a pharmacological agent that depolymerizes actin (e.g. latrunculin). Suitable macropinocytosis inhibitors are able to be determined by one skilled in the art. Suitable micropinocytosis inhibitors include, for example, imipramine (3-(10,11-dihydro-5H-dibenzo[b,f]azepin-5-yl)-N,N-dimethylpropan-1-amine), phenoxybenzamine ((RS)—N-Benzyl-N-(2-chloroethyl)-1-phenoxypropan-2-amine) and vinblastine (dimethyl (2β, 3β, 4β, 5α, 12β, 19α)-15-[(5S,9S)-5-ethyl-5-hydroxy-9-(methoxycarbonyl)-1,4,5,6,7,8,9,10-octahydro-2H-3,7-methanoazacycloundecino[5,4-b]indol-9-yl]-3-hydroxy-16-methoxy-1-methyl-6,7-didehydroaspidospermidine-3,4-dicarboxylate), amiloride hydrochloride, wortmannin, latrunculin B (Lat B) among others. In some embodiments, the macropinocytosis inhibitor is latrunculin A (LatA) or LatB.

As used herein, the term “effector nanoparticle” refers to a nanoparticle employed for either diagnostic or therapeutic applications that will be enhanced by decreased MPS clearance. In some embodiments, the effector nanoparticle is a micelle, bicontinuous nanosphere, vesicular polymersomes (PS), filamentous worm-like micelles, among others. Suitable effector nanoparticles would be understood by one skilled in the art, including, for example, liposomes and lipid nanoparticles (LNPs). LNPs are spherical vesicles made of ionizable lipids that are taken up by endocytosis, and can include helper lipids (promote cell binding), cholesterol to fill the gaps between the lipids, and polyethylene glycol (PEG) to reduce opsonization The term lipid is used to include known lipids, including, for example, triglycerides (e.g. tristearin), diglycerides (e.g. glycerol bahenate), monoglycerides (e.g. glycerol monostearate), fatty acids (e.g. stearic acid), steroids (e.g. cholesterol), and waxes (e.g. cetyl palmitate), among others. All classes of emulsifiers can been used to stabilize the lipid dispersion.

In some embodiments, the effector nanoparticle comprises a targeting moiety incorporated into the surface of the nanoparticle. As used herein, the term “targeting moiety” refers to compound, polypeptide or molecules that could be stably retained within self-assembled PEG-b-PPS nanoparticles for controlled surface display. The targeting moiety allows for the targeting of the nanoparticle to specific cells within a system, e.g., within a subject.

In some embodiments, the targeting moiety comprises a cell-receptor binding molecule. In some embodiments, the targeting moiety comprises (a) a cell-receptor-binding molecule, (b) alinker, e.g. a polyethylene glycol (PEG) linker, and (c) an anchor.

As used herein, the term “cell-receptor-binding molecule” refers to a molecule that is capable of binding to a specific receptor expressed on the surface of targeted cells. Suitable cell-receptor binding molecules, include, for example, peptides, proteins/glycoproteins (for example antibodies and transferrin), aptamers, lipids and small molecules like folate. These molecules are known in the art, including, for example, those described in Roland Böttger et. al., Lipid-based nanoparticle technologies for liver targeting, Advanced Drug Delivery Reviews, Volumes 154-155, 2020, Pages 79-101, ISSN 0169-409X, //doi.org/10.1016/j.addr.2020.06.017., incorporated by reference in its entirety. In some embodiments, targeting can be achieved by controlling protein adsorption and surface chemistry of the nanoparticles, as demonstrated in Vincent, M. P., Bobbala, S., Karabin, N. B. et al. Surface chemistry-mediated modulation of adsorbed albumin folding state specifies nanocarrier clearance by distinct macrophage subsets. Nat Commun 12, 648 (2021). doi.org/10.1038/s41467-020-20886-7 and Vincent, M. P., Karabin, N. B., Allen, S. D., Bobbala, S., Frey, M. A., Yi, S., Yang, Y. and Scott, E. A. (2021), The Combination of Morphology and Surface Chemistry Defines the Immunological Identity of Nanocarriers in Human Blood. Adv. Therap. 2100062.//doi.org/10.1002/adtp.202100062, the contents of which are incorporated by reference in their entireties regarding protein adsorption and surface chemistry of the nanoparticles to control targeting.

In some embodiments, the molecule is capable of binding to receptors expressed on tumors. In some embodiments, the cell-receptor-binding molecule is folate.

As used herein, the term “linker” or “spacer” refers to molecules or short amino acid sequences that separate multiple domains, e.g., the targeting moiety from the region, e.g., anchor, that attaches it to the nanoparticle surface. The linkers are suitable flexible to allow the proper display of the targeting moiety. The linker may be a short amino acid sequences, e.g., any amino acid sequences, but can preferably be glycine or alanine amin acids. Any suitable linkers known in the art can be used.

In another embodiment, the linker is a PEG linker. Suitably, a “PEG linker” is a linker of PEG having from about 6 to 48 units. A unit of PEG comprises —(O—CH₂—CH₂)— and in some embodiments the PEG linker has a formula “—HN—C(O)—CH₂—(O—CH₂—CH₂)ₙ—NH—C(O)—.” In some embodiments, one end is attached to the cell-receptor-binding molecule and the other end is attached to the carboxylic acid lipid anchor, and “n” is an integer of 6-48. In some embodiments, n is 23.

As used herein, the term “anchor” refers to a lipid anchor that can be embedded into the surface of the nanoparticle. In one example, the anchor is a carboxylic acid lipid anchor that is attached to the PEG linker and partitions into the hydrophobic PPS core of the effector nanoparticle. In some embodiments, the anchor is a palmitoleic acid anchor. Other suitable anchors are known and understood in the art.

In some embodiments, the effector nanoparticle further comprises a therapeutic payload. As used herein, the term “therapeutic payload” refers to a molecule or drug that is capable of treating a disease or condition. In preferred embodiments, the therapeutic payload is an agent capable of treating cancers, e.g., anti-cancer chemotherapeutics. The therapeutic agent are able to be determined by one skilled in the art and are readily known.

The formulations disclosed herein may also be incorporated into pharmaceutical compositions. The disclosed formulations or pharmaceutical compositions comprising the same may be used in methods for enhancing circulation time and/or cell-targeting efficacy of an effector nanoparticle in a subject. In some embodiments, the pharmaceutical compositions may further comprise one or more pharmaceutically acceptable carriers.

In some embodiments, the pharmaceutical compositions may further comprise one or more nanoparticles capable of presenting cell-receptor-binding molecules on their surfaces, in particular self-assembled systems like amphiphilic block copolymers other than PEG-b-PPS (e.g. PEG-PLGA) or systems capable of using similar targeting strategies to reach specific cells for the treatment of specific diseases, such as cancer.

The pharmaceutical compositions used in the methods of the present invention may be formulated in any form that is appropriate for administration to the subject. For example, one or more of the agents may be formulated with a pharmaceutically acceptable carrier. The term “pharmaceutically acceptable carrier” refers to any carrier, diluent or excipient that is compatible with the other ingredients of the formulation and not deleterious to the recipient. Preferably, the pharmaceutically acceptable carrier is chosen in accordance with the selected route of administration and standard pharmaceutical practice for each agent. For example, for oral administration, the active ingredient may be combined with one or more inactive ingredients for the preparations. The agents used in the methods of the present invention may be formulated into dosage forms according to standard practices in the field of pharmaceutical preparations. See, e.g., Alphonso Gennaro, ed., Remington's Pharmaceutical Sciences, 18th Ed., (1990) Mack Publishing Co., Easton, Pa.

The amount of the disclosed pharmaceutical compositions comprising the formulation to be administered is dependent on a variety of factors, including the severity of the condition, the frequency of administration, the duration of treatment, and the like. The disclosed pharmaceutical compositions may be administered at any suitable dosage, frequency, and for any suitable duration necessary to achieve the desired therapeutic effect.

The disclosed pharmaceutical compositions may be administered once per day or multiple times per day. Alternatively, the pharmaceutical compositions may be administered once per week. In other examples, the pharmaceutical compositions may be administered once per day, twice per day, or three or more times per day. The disclosed pharmaceutical compositions may be administered daily, every other day, every three days, every four days, every five days, every six days, once per week, once every two weeks, or less than once every two weeks. The pharmaceutical compositions may be administered for any suitable duration to achieve the desired therapeutic effect. For example, the pharmaceutical compositions may be administered to the subject for one day, two days, three days, four days, five days, six days, seven days, eight days, nine days, ten days, eleven days, twelve days, thirteen days, two weeks, one month, two months, three months, six months, 1 year, or more than 1 year.

Any suitable dose of the disclosed pharmaceutical compositions comprising the formulation may be used. Suitable doses will depend on the therapeutic agent, intended therapeutic effect, body weight of the individual, age of the individual, and the like.

In another embodiment, the dosage of polymer may depend on how much drug is loaded, and may range from low levels (e.g., <1 μg/mL) for high loading of LatA to high levels (e.g., >200 mg/mL) for low loading of LatA within particles, and includes ranges in between. In some embodiments, the suitable dose of LatA is from 0.1 mg/kg to 1.0 mg/kg, or from 0.15 mg/kg to 0.95 mg/kg, or from 0.2 mg/kg to 0.9 mg/kg, or from 0.25 mg/kg to 0.85 mg/kg, or from 0.3 mg/kg to 0.8 mg/kg, or from 0.35 mg/kg to 0.75 mg/kg, or from 0.4 mg/kg to 0.7 mg/kg, or from 0.45 mg/kg to 0.65 mg/kg, or from 0.5 mg/kg to 0.6 mg/kg. In some embodiments, the suitable dose of LatA is 0.55 mg/kg.

Methods

The present disclosure also provides in some embodiments methods for enhancing circulation time and/or cell-targeting efficacy of an effector nanoparticle in a subject. In some embodiments, the method comprises administering any one of the formulations as described herein.

Suitable modes of administration include, without limitation, subcutaneous administration and intravenous administration. In some embodiments, the formulations as described herein or pharmaceutical compositions comprising the same is administered subcutaneously to a subject. In some embodiments, subcutaneous administration of the formulation or pharmaceutical composition comprising the same allows effector nanoparticles to achieve serum levels that rival a standard intravenous administration. In some embodiments, the administering is performed subcutaneously and accumulation of the effector nanoparticle in serum of the subject is increased.

The term “subject” are used herein interchangeably to refer to a mammal, preferably a human, to be treated by the methods, formulations, and compositions described herein. “Mammals” means any member of the class Mammalia including, but not limited to, humans, nonhuman primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Preferably, the subject is a human. In some embodiments, the subject is a mammal in need of gene therapy. The term “subject” does not denote a particular age or sex. In one specific embodiment, a subject is a mammal, preferably a human. In a suitable embodiment, the subject is a human in need of reduction of IOP. In another embodiment, the subject is a subject having one or more symptom of glaucoma.

In some embodiments, uptake of the effector nanoparticle by the mononuclear phagocyte system (MPS) is inhibited by administering the formulations as described herein or pharmaceutical compositions comprising the same. As used herein, the term “mononuclear phagocyte system” refers to a diverse population of phagocytic cells primarily located within the liver, spleen, and lymph nodes. The MPS includes monocytes, macrophages/histiocytes, and dendritic cells. These cells function in tissue repair and remodeling, resolution of inflammation, maintenance of homeostasis and to clear foreign bodies for the system. This system indiscriminately clears nanoparticles from circulation via receptor-dependent and independent mechanisms of endocytosis, inducing side effects and decreasing efficacy.

In some embodiments, receptor-mediated endocytosis in a subject is not inhibited by administration of the formulations as described herein or pharmaceutical compositions comprising the same. As used herein, the term “receptor-mediated endocytosis” refers to a process by which cells absorb metabolites, hormones, proteins, and molecules, by the inward budding of the plasma membrane (invagination).

In some embodiments, the cell-receptor-binding molecule in the targeting moiety incorporated into the surface of the effector nanoparticle binds to tumor cells. In some embodiments, the tumor is melanoma.

In some embodiments, the uptake of the effector nanoparticles by the targeted cells is increased by at least 1-fold. In some embodiments, the uptake of the effector nanoparticles by the targeted cells is increased by at least 2-fold, or at least 3-fold, or at least 4-fold, or at least 5-fold, or at least 6-fold, or at least 7-fold, or at least 8-fold.

Despite numerous advancements in strategies for targeting specific cells, little to no progress has been made regarding the prevention of nonspecific clearance of nanoparticles by the mononuclear phagocyte system (MPS), which remains a key limitation of precision drug delivery. A median average of only 0.7% of administered nanoparticles successfully reaches solid tumors during cancer nanotherapy despite the use of surface-conjugated targeting moieties like antibodies, peptides, or aptamers. Furthermore, both protein adsorption and the numbers and/or activation state of MPS cells depend on patient-specific biochemistry and inflammation, which vary by disease state. The present invention addresses this issue by reducing the nanoparticle clearance to improve therapeutic targeting of cells within tumors.

The present invention describes a novel multi-nanoparticle strategy that safely and temporarily inhibits macropinocytosis by MPS cells, while allowing receptor-mediated cellular targeting to continue unimpeded as described in the Examples. In this method, macropinocytosis inhibitory nanoparticles (MiNP) are pre- or co-administered with “effector” nanoparticles (E-NP), the latter of which can perform either a diagnostic or therapeutic function. This “indirect” targeting (e.g. modulation of off-target cells by MiNP) enhances the circulation time, serum concentration, and thus targeting of E-NP without needing to directly modify the E-NP. This method is distinct from and complements known “active” and “passive” targeting strategies, which respectively employ targeting ligands or specific nanoparticle physicochemical properties for cell-selective uptake.

As demonstrated in the examples, MiNP contain macropinocytosis inhibitor latrunculin A (LatA), a transient actin depolymerizing agent which is hydrophobic and thus not amenable to direct administration via subcutaneous (s.c.) or intravenous (i.v.) routes. As demonstrated in the examples, MiNP temporarily inhibit the non-specific MPS uptake of a subsequent chasing dose of an E-NP by increasing blood concentration and tumor accumulation compared to E-NP administered alone. Of note, this MiNP co-administration strategy significantly improved the s.c. injection of E-NP, achieving systemic bioavailability for E-NP on par with i.v. injections.

The present disclosure also provides in some embodiments methods for enhancing circulation time and/or cell-targeting efficacy of an effector nanoparticle in a subject. In some embodiments, the method comprises the steps of (1) administering a macropinocytosis inhibitory nanoparticle (MiNP) and (2) administering an effector nanoparticle. In some embodiments, the macropinocytosis inhibitory nanoparticle comprises (a) a nanostructure (e.g., micelles or BCN, among others) comprising a poly(ethylene glycol)-block-poly(propylene sulfide) (PEG-b-PPS) copolymer and (b) a macropinocytosis inhibitor loaded in the nanostructure.

In some embodiments, the MiNP is administered prior to the effector nanoparticle (the pre-injection dosing method). This pre-injection results in the temporary inhibit the non-specific MPS uptake allowing the E-NP to be delivered and exert their effect before being cleared from the system.

In some embodiments, the MiNP is administered simultaneously with the effector nanoparticle (the co-injection dosing method). Co-injection was shown to provide the benefit of temporary inhibit the non-specific MPS uptake allowing the E-NP to be delivered and exert their effect before being cleared from the system.

In some embodiments, the co-injection dosing method is equivalent to the pre-injection dosing method regarding the amount of the reduced uptake of the effector nanoparticle by MPS cells. In some embodiments, the co-injection dosing method reduces same or similar amount of uptake of the effector nanoparticle by MPS cells as the pre-injection dosing method. In some embodiments, the MPS cells are in the spleen of a subject. In other embodiments, the MPS cells are in the liver of the subject.

In some embodiments, the co-injection dosing method is superior to the pre-injection dosing method regarding the amount of the reduced uptake of the effector nanoparticle by MPS cells. In some embodiments, the co-injection dosing method reduces more uptake of the effector nanoparticle by MPS cells. In some embodiments, the MPS cells are in the liver of a subject.

In some embodiments, MOS phagocytes are affected by the co-injection dosing method at a greater or equivalent level than the pre-injection dosing method.

The use herein of the terms “including,” “comprising,” or “having,” and variations thereof, is meant to encompass the elements listed thereafter and equivalents thereof as well as additional elements. Embodiments recited as “including,” “comprising” or “having” certain elements are also contemplated as “consisting essentially of” and “consisting of” those certain elements.

As used herein, “about” means within 5-10% of a stated concentration range or within 5-10% of a stated number.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit's interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The present invention has been described in terms of one or more preferred embodiments, and it should be appreciated that many equivalents, alternatives, variations, and modifications, aside from those expressly stated, are possible and within the scope of the invention.

The following Examples are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### Example 1

Reference is made to the article “Enhancing subcutaneous injection and target tissue accumulation of nanoparticles via co-administration with macropinocytosis inhibitory nanoparticles (MiNP),” Nanoscale Horiz, 2021, 6, 393, the content of which is incorporated by reference in entirety.

A significant barrier to the application of nanoparticles for precision medicine is the mononuclear phagocyte system (MPS), a diverse population of phagocytic cells primarily located within the liver, spleen and lymph nodes. The majority of nanoparticles are indiscriminately cleared by the MPS via macropinocytosis before reaching their intended targets, resulting in side effects and decreased efficacy. Here, we demonstrate that the biodistribution and desired tissue accumulation of targeted nanoparticles can be significantly enhanced by co-injection with polymeric micelles containing the actin depolymerizing agent latrunculin A. These macropinocytosis inhibitory nanoparticles (MiNP) were found to selectively inhibit non-specific uptake of a second “effector” nanoparticle in vitro without impeding receptor-mediated endocytosis. In tumor bearing mice, co-injection with MiNP in a single multi-nanoparticle formulation significantly increased the accumulation of folate-receptor targeted nanoparticles within tumors. Furthermore, subcutaneous co-administration with MiNP allowed effector nanoparticles to achieve serum levels that rivaled a standard intravenous injection. This effect was only observed if the effector nanoparticles were injected within 24 h following MiNP administration, indicating a temporary avoidance of MPS cells. Co-injection with MiNP therefore allows reversible evasion of the MPS for targeted nanoparticles and presents a previously unexplored method of modulating and improving nanoparticle biodistribution following subcutaneous administration.

New Concepts

Precision nanoparticle therapeutics aim to deliver a maximum therapeutic payload to tissues of interest while minimizing off target effects. Non-specific clearance of these nanoparticles by cells of the mononuclear phagocyte system (MPS) reduces accumulation of nanoparticles in target tissues and thereby efficacy. Herein, we demonstrate the development of novel macropinocytosis inhibitory nanoparticles (MiNP) which are able to reduce non-specific MPS clearance of co-administered “effector-NPs”. MiNP are able to increase the tumor accumulation of effector-NPs in a mouse tumor model as well as increase serum accumulation of subcutaneously injected effector-NPs. These biodistribution altering effects were only seen for 24 h after MiNP administration, indicating the transient nature of this system. Combinatorial approaches are needed in order to maximize evasion of the MPS, and this adaptable, modular system could be combined with other “stealth” nanoparticle strategies to create synergistic effects. This type of transient modular platform system for MPS evasion has not been demonstrated previously. Our approach leads to a system which can be adapted to a variety of practical “effector-NPs” in order to increase their target tissue and serum accumulation, thereby enhancing existing and in-development nanoparticle therapeutics.

Nanoparticles are versatile carriers that can improve and often specify the stability, circulation time, and biodistribution of therapeutic molecules.¹ Despite these advantages, rapid clearance of nanoparticles by the mononuclear phagocyte system²(MPS) remains a significant barrier to their applications in precision medicine. The MPS consists of circulating and organ-resident phagocytic cells, which internalize nanoparticles and eventually clear them through the liver.³⁻⁶ A comprehensive survey of the literature reported that a median average of only 0.7% of administered nanoparticles successfully reach solid tumors despite the use of surface-conjugated targeting moieties like antibodies, peptides or aptamers.⁷ Clearance by MPS cells occurs primarily in the liver, spleen and lymph nodes through a number of endocytic pathways including clathrin-mediated and clathrin-independent pathways, macropinocytosis, and phagocytosis.⁸ Macropinocytosis is a process by which membranes extend and form around extracellular fluid leading to internalization of the encapsulated region, while phagocytosis is usually receptor-initiated and internalizes with or without extension of plasma membranes through the use of membrane invaginations.⁹ If these pathways are temporarily inhibited in MPS cells prior to or in conjunction with the introduction of therapeutic nanoparticles, the bioavailability and therapeutic efficacy of these nanoparticles would likely increase significantly.¹⁰ Developing nanoparticles with “stealth” properties to avoid this non-specific uptake remains a critical objective for nanomedicine and many different strategies such as PEGylation and CD47 “don't eat me” peptides, have been tried with variable levels of effectiveness.¹¹⁻¹³ Combinatorial strategies employing multiple different stealth strategies are needed to further reduce clearance by the MPS and increase nanomaterial utility in vivo.

We have previously demonstrated that diverse nanoparticle morphologies can be self-assembled from poly(ethylene glycol)-block-poly(propylene sulfide) (PEG-b-PPS) copolymers to function as customizable and non-toxic drug delivery vehicles.^{14,15} The nanostructure morphology and route of administration dictate the biodistribution of PEG-b-PPS nanoparticles, allowing the passive and preferential targeting of different phagocytic cell populations in vivo without the need for surface-conjugated targeting ligands.^{14,16-19} Of note, spherical solid core PEG-b-PPS micelles are primarily taken up by liver macrophages following intravenous (IV) administration¹⁷ and monocyte populations in draining lymph nodes and spleen following subcutaneous (SC) administration,¹⁶ both of which are key components of the MPS.²⁰ At time points of 24 h and less, nanoparticles smaller than 100 nm in diameter primarily reach lymphoid organs directly²¹ as up to 48 h is typically required for trafficking of peripheral phagocytes to these locations.²² We have previously verified SC injected PEG-b-PPS micelles to reach the spleen intact after filtering through draining lymph nodes.^{21,23} These micelles have also been previously shown to reduce the cytotoxicity of small molecule drugs such as Celastrol.²⁴ Here we employ macropinocytosis inhibitory nanoparticles (MiNP) to reduce specific nanoparticle uptake by the MPS and enhance their accumulation within target tissues. MiNP are comprised of PEG-b-PPS micelles containing Latrunculin A (LatA), a well-known and transient actin depolymerizing agent.²⁵ LatA is most commonly used to temporarily inhibit macropinocytosis²⁶ by phagocytic cells during in vitro assays to investigate mechanisms of cell endocytosis. Furthermore, LatA is hydrophobic and thus not amenable to direct administration via SC or IV routes. We have previously shown that LatA retains its inhibitory effects by disrupting the cell cytoskeleton when encapsulated in PEG-b-PPS micelles but without toxicity.^{27,28}

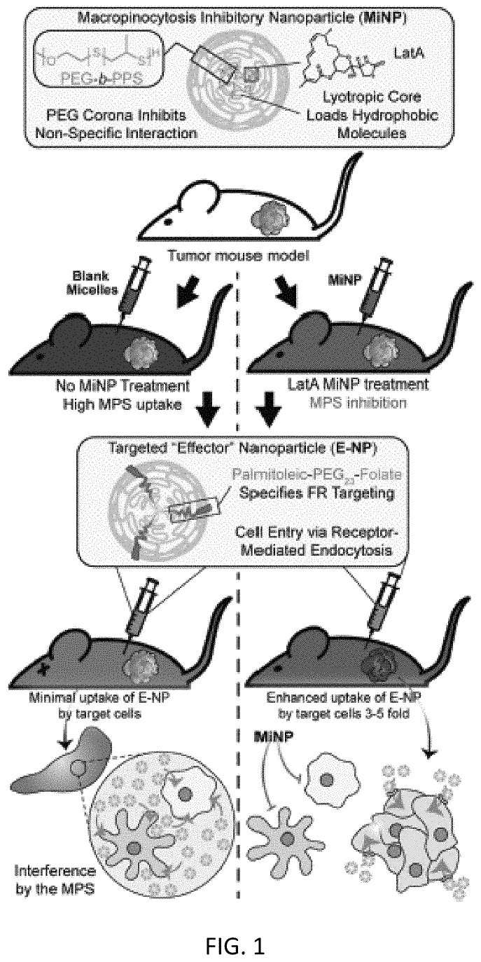

We selected to investigate and optimize a co-administration strategy wherein MiNP are injected before and/or simultaneously with a model “effector” nanoparticle, which represents a nanoparticle employed for either diagnostic or therapeutic applications that will be enhanced by decreased MIPS clearance. For the purposes of this proof of concept study, the employed model effector nanoparticle is a fluorescent PEG-b-PPS micelle (E-MC). The inhibitory effects of MiNP were characterized in vitro using macrophages and in vivo in a B16F10 melanoma tumor bearing mouse model. Furthermore, E-MC with surface-decorated folate (E-MC(FA)) were used to explore the ability of MiNP to enhance the accumulation of a targeted E-MC within folate receptor-expressing solid tumors (FIG. 1). We find that IV or SC injection of MiNP temporarily inhibit the non-specific MPS uptake of a subsequent chasing dose of an E-MC by increasing blood concentration and tumor accumulation compared to E-MC administered alone. Of note, this MiNP co-administration strategy significantly improved the SC injection of E-MC, achieving bioavailability of E-MC on par with IV injections.

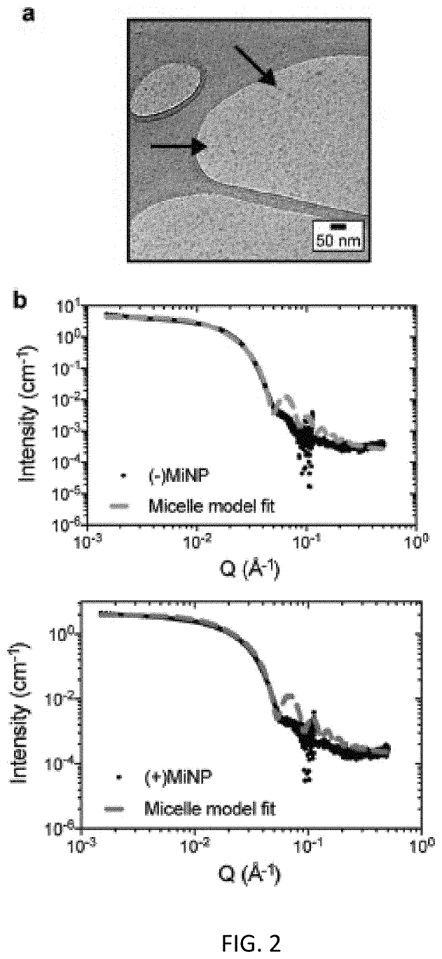

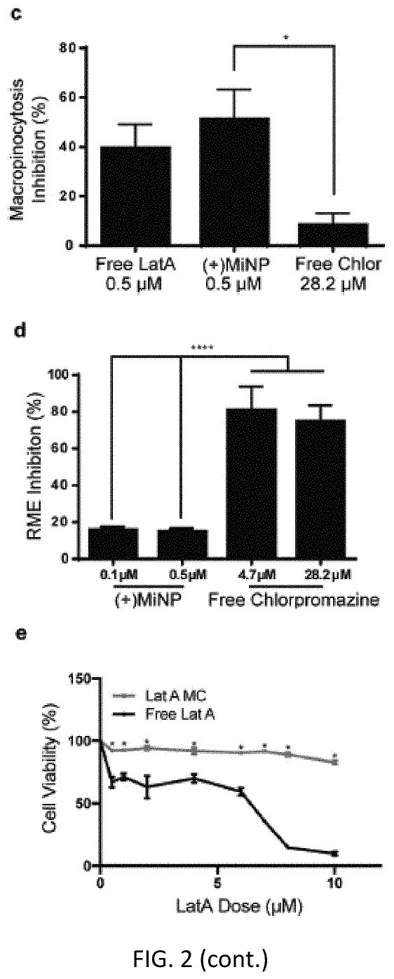

LatA-loaded ((+) MiNP) and unloaded controls ((−) MiNP) were self-assembled from PEG₄₅-b-PPS₂₃ copolymer using the co-solvent evaporation method.²⁹ Dynamic light scattering was used to determine the z-average and polydispersity of the different formulations (Table 1, ESI†). Confirmation of the micelle structure was obtained using cryogenic transmission electron microscopy and small angle X-ray scattering (SAXS) studies (FIGS. 2a and b). Small angle X-ray scattering curves of both (+) MiNP and (−) MiNP were successfully fitted with a micelle model indicating retention of micellar nanostructures for (+) MiNP after loading with LatA (FIG. 2b). The core radius and approximate diameter of both (−) MiNP and (+) MiNP obtained using SAXS model fits are reported in Table 2 (ESI†). LatA quantification and loading within the micelles was determined by HPLC-UV as previously reported²⁸ and allowed for all formulations to be referenced based on their LatA content (Table 1, ESI†). These data are consistent with our previous findings that encapsulation of LatA by PEG-b-PPS micelles does not alter their physical structure or polydispersity.

TABLE 1
Size and encapsulation efficiency (EE) of MiNP as determined by
Dynamic Light Scattering (DLS) and HPLC. Data are presented for unloaded PEG-b-PPS micelles
((−)MiNP) and latrunculin A (LatA) loaded PEG-b-PPS micelles ((+)MiNP.

| | Number Avg (nm) | PDI | LatA EE |
|---|---|---|---|
| (−)MiNP | 22.8 ± 4.65 | 0.08 | N/A |
| (+)MiNP | 20.1 ± 5.36 | 0.07 | 61.5% |

TABLE 2
Physical characteristics of MiNP as determined by small angle x-
ray scattering (SAXS). Data are presented for
unloaded ((−)MiNP) and latrunculin A
loaded ((+)MiNP) micelles. All values including radius of gyration
(Rg) were obtained using SAXS model fits.

| | (−)MiNP | (+)MiNP |
|---|---|---|
| Radius of Core (nm) | 8.9 | 8.3 |
| Rg (nm) | 7.1 | 7.03 |
| Diameter | 32 | 30.6 |
| Chi² | 0.005 | 0.008 |

To investigate macropinocytosis inhibition by MiNP and determine whether this inhibition still permits uptake via receptor-mediated endocytosis, (+) MiNP were compared to chlorpromazine, an inhibitor of receptor-mediated endocytosis. These distinct mechanisms of endocytosis were evaluated using dextran conjugated pHrodo dye and transferrin conjugated pHrodo dye to respectively quantify effects of (+) MiNP and chlorpromazine on macropinocytosis (dextran) and receptor-mediated endocytosis (transferrin). Free LatA, (+) MiNP, and free chlorpromazine were incubated with RAW264.7 macrophages for 2 hours and subsequently washed and then chased with dextran-pHrodo (FIG. 2c) or transferrin-pHrodo (FIG. 2d). After 30 minutes of incubation, the cells were harvested and analyzed by flow cytometry to quantify and compare uptake of dextran-pHrodo via macropinocytosis and transferrin-pHrodo by transferrin-receptors. In the case of dextran (macropinocytosis), (+) MiNP and free LatA both showed much stronger inhibition of uptake than free chlorpromazine (FIG. 2c). In the case of transferrin (receptor mediated endocytosis), free chlorpromazine at both high and low doses significantly inhibited endocytosis compared to (+) MiNP, which had minimal impact (FIG. 2d). These results demonstrate that the functional aspect of LatA is not significantly altered by encapsulation in PEG-b-PPS micelles. Importantly, MiNP did not impede uptake of transferrin via transferrin receptors, suggesting that MiNP could be employed in a multi-nanoparticle strategy to inhibit non-specific uptake of a targeted chase nanoparticle while simultaneously permitting receptor-mediated targeting of specific cell populations. As LatA has been shown to be cytotoxic at higher doses and has been used as a cytotoxic agent,^{30,31} we sought to evaluate the cytotoxicity of MiNP on our target cell population of macrophages. After 2 hours of exposure to various doses of (+) MiNP and Free LatA, (+) MiNP treated macrophages remained highly viable at all tested concentrations, while free LatA treated macrophages demonstrated significant toxicity at doses of 0.5 μM and above (FIG. 2e). This is consistent with our previous findings in which encapsulation of small molecule drug Celastrol reduced its cytotoxicity in vitro.²⁴

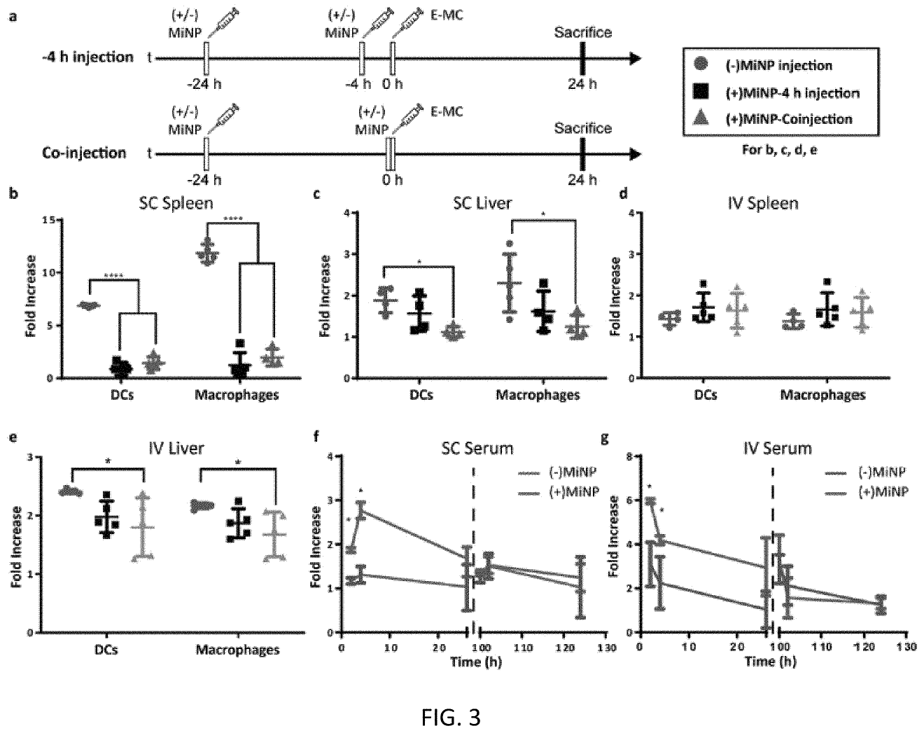

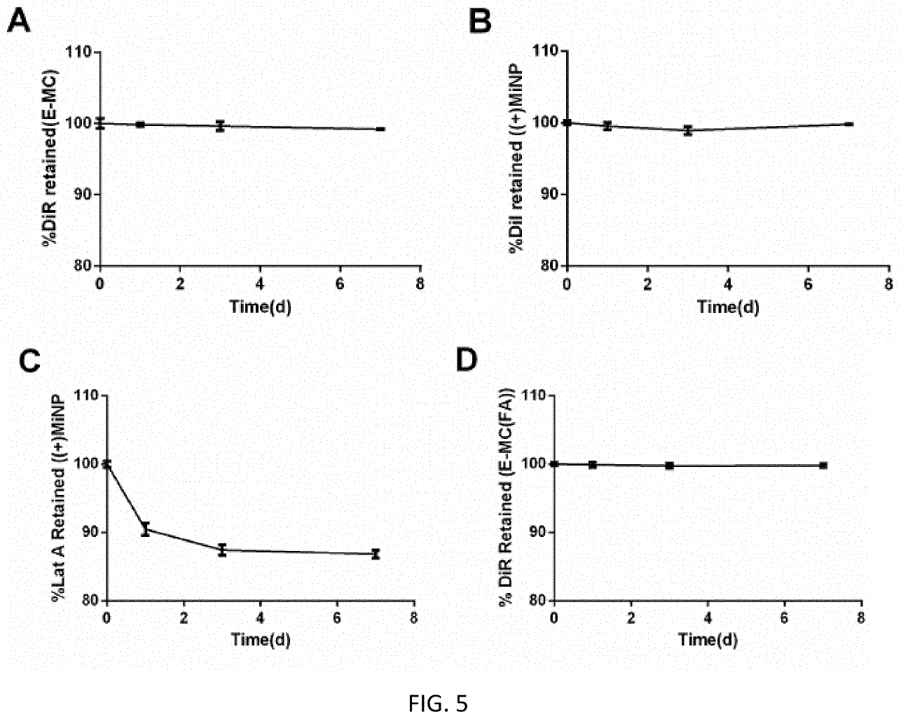

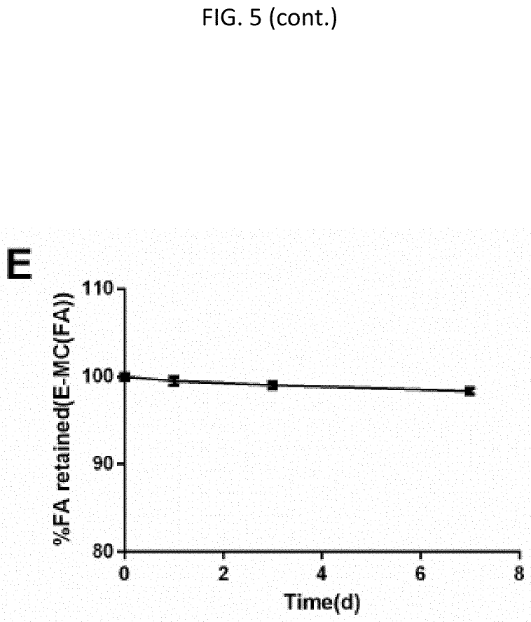

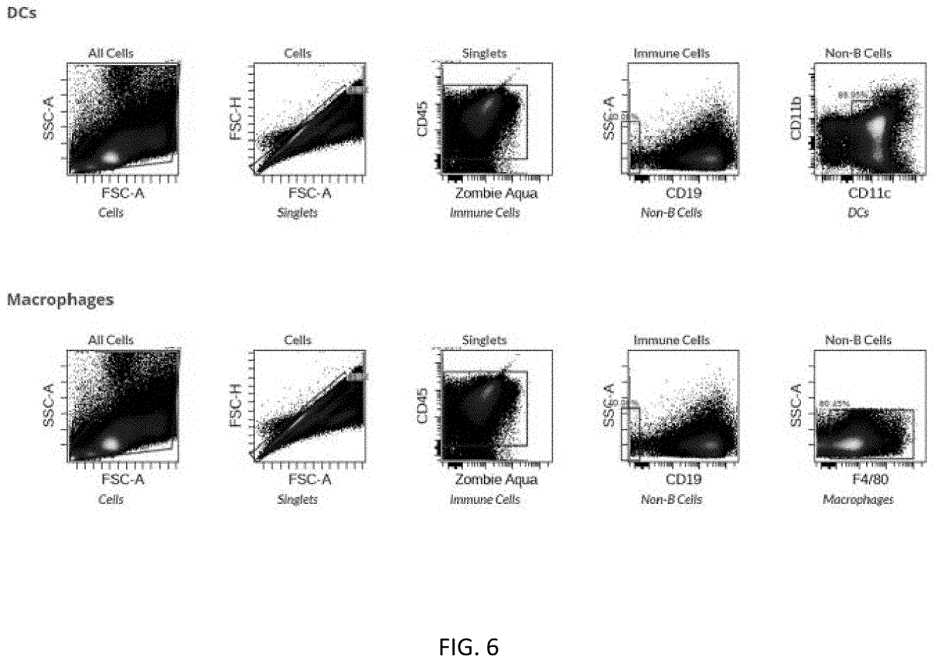

Having characterized MiNP in vitro, we next investigated different in vivo dosing regimens to evaluate the effect of MiNP on the uptake and biodistribution of a subsequently injected blank effector nanoparticle (E-MC) without targeting moieties (FIG. 3a). Importantly, we sought to determine whether (+) MiNP could be administered SC with the E-MC in the same formulation versus administered as a separate injection prior to administration of the E-MC. For quantification of cell uptake, E-MC were labelled with DiR dye. We have previously demonstrated that lipophilic dyes are stably retained within PEG-b-PPS nanocarriers for in vivo applications and flow cytometric analysis.^{15,23,32} We further confirmed the stability of DiI, DiR and LatA within E-MC and (+) MiNP over the course of 7 days using membrane dialysis (FIG. 5A-C, ESI†). First, a pre-injection strategy was tested where (±) MiNP were administered twice at both 24 h and 4 h prior to injection of the chase nanoparticle. As we have previously published that the height of PEG-b-PPS micelle uptake by the MIPS occurs at the 24 h timepoint,¹⁷ we suspected that this regime would extensively pre-condition and shut down the MIPS to avoid non-specific clearance of the E-MC. A simplified procedure was also evaluated wherein the (±) MiNP were administered alone just once to pre-condition the mouse, which was then followed 24 h later by a co-injected dose of a (±) MiNP and E-MC multi-nanoparticle formulation (FIG. 3a). In both regimens, the same total micelle dosage of (+) MiNP or (−) MiNP and E-MC were administered. The total dosage of (−) MiNP and E-MC administered was equal to a LatA dose of 100 μL of 7 μM (+) MiNP solution (approx. 0.55 mg kg⁻¹). This dosage was based upon previously reported intraperitoneal treatments of mice³⁰ and our in vitro toxicity assessment in RAW264.7 macrophages (FIG. 2e). Mice were sacrificed 24 h after the chase injection, and organs were harvested for analysis by flow cytometry. Dendritic cells (DCs) and macrophages, the two key phagocytes of the MPS, were identified via antibody staining and the amount of E-MC in each cell population was quantified (FIG. 6, ESI†). In the spleen, (+) MiNP treatment with both regimes showed significantly less E-MC uptake by MPS cells than mice injected with (−) MiNP (FIG. 3b). In the liver, the multi-nanoparticle co-injection (+) MiNP/E-MC formulation had significantly less chase particle uptake than (−) MiNP/E-MC in all cell types, while the 4 h pre-injection regimen did not. These results verify that a MiNP strategy indeed inhibits uptake of a second effector nanoparticle in both the spleen and liver following SC injection. Furthermore, this indicates that in both organs, MPS phagocytes are affected by the multi-nanoparticle (+) MiNP/E-MC co-injection dosing method at a greater or equivalent level than the 4 h separate pre-injection method. The finding that the −4 h and co-injection of MiNP were equally effective, provides some indication of the time scale of the effect of the LatA-loaded nanoparticles. This suggests that the immediate effect of LatA occurs on the same time scale as the E-MC uptake, begins to wane within 4 h, and persists for at least 24 h. Additionally, the co-injection method is simpler to administer and would be preferred for any future translation of this system. As such, the co-injection multi-nanoparticle method was deemed superior and was the method of choice for future experiments. We intend to further address and optimize the MiNP injection schedule in future studies.

We next sought to compare the effects of MiNP following SC versus IV administration. A majority of nanotherapeutics are administered IV out of necessity, as nanoparticles are rapidly cleared by phagocytes during lymphatic drainage. As IV administration must be performed by healthcare professionals, enhancing SC administration to achieve IV-level biodistribution of nanoparticles would permit facile administration and more flexible dose schedules, possibly increasing patient compliance and access to treatment.

Similar to SC injections, the co-injection and −4 h injection methods were followed for IV administration and the dose remained consistent at 100 μL of 7 μM LatA (+) MiNP. IV injection had a distinctly different uptake profile than SC injections, demonstrating no difference in E-MC uptake in the spleen with either injection method (FIG. 3d). However, in the liver, treatment with (+) MiNP decreased uptake of E-MC in DC and macrophage populations when administered via the co-injection method, but not the −4 h method, indicating altered biodistribution within two MPS cell types of interest (FIG. 3e). These data further confirm the co-injection method to be equivalent or superior to the −4 h injection method.

We next evaluated serum levels of chase nanoparticles after SC and IV administration of (+) MiNP. LatA inhibits actin polymerization by binding actin at a 1:1 ratio and consuming intracellular LatA.³³ Thus, its effects should decrease over time due to continuous LatA depletion without replenishment. We therefore investigated the transient effects of (+) MiNP by evaluating whether the inhibitor's effects would diminish within 100 h of the initial injection. Mice were divided into a (+) MiNP group that was administered a (+) MiNP/E-MC co-injection and a (−) MiNP control group that was administered (−) MiNP/E-MC.

Blood (100 μL) was collected from each mouse at 2 h, 4 h and 26 h post SC or IV co-injection, and serum was isolated and analysed to assess E-MC content by spectrophotometry. Following final blood collection at 26 h, mice were rested for 74 h and subsequently injected with E-MC a second time at 100 h to assess any residual effects of the original MiNP administration. Blood (100 μL) was again collected from mice at 102 h, 104 h, and 126 h relative to the initial MiNP administration to quantify E-MC content by spectrophotometry. (+) MiNP treatment increased E-MC content in serum at 2 h and 4 h post injection following both IV and SC administration. The SC injections resulted in a delay in reaching the maximum serum level of the chase, which occurred at 4 h and is indicative of the time required for the nanoparticles to drain from the SC tissue and reach systemic circulation. After the mice were rested, there was no difference in the E-MC serum content between mice administered (+) MiNP or (−) MiNP after the second chase injection (FIGS. 3f and g). This indicates the mice returned to a baseline processing of E-MC by 100 h post (+) MiNP treatment for both routes of administration. Another interesting observation from this data was that the (+) MiNP SC injection group had a similar E-MC serum content at 4 h as the (−) MiNP IV injection group, suggesting that (+) MiNP administered subcutaneously are able to achieve a similar amount of E-MC serum concentration as the typically used IV injection ((−) MiNP treatment). Upon observing this, we measured the total area under the curve over the course of the first 26 h and found that SC (+) MiNP treatment had a value of 53.5 h, an increase over the IV (−) MiNP value of 41.2 h (Table 3, ESI†). This further confirms the enhanced serum levels of E-MC in response to SC injection of (+) MiNP. This effect in conjunction with a therapeutic payload would allow access to a host of different dosing strategies for existing nanotherapeutics as well as easier administration.

TABLE 3
Area under the curve (AUC) from 0-26 hours post injection. Following
subcutaneous (SC) or intravenous (IV) injections with (+/−)MiNP and E-MC, mouse serum
content of E-MC was measured over the next 26 hours. AUC was calculated using GraphPad
Prism software.

| Group | AUC 0-26 h (+/−)MiNP injections |
|---|---|
| SC (−)MiNP | 28.28 |
| SC (+)MiNP | 53.51 |
| IV (−)MiNP | 41.24 |
| IV (+)MiNP | 88.39 |

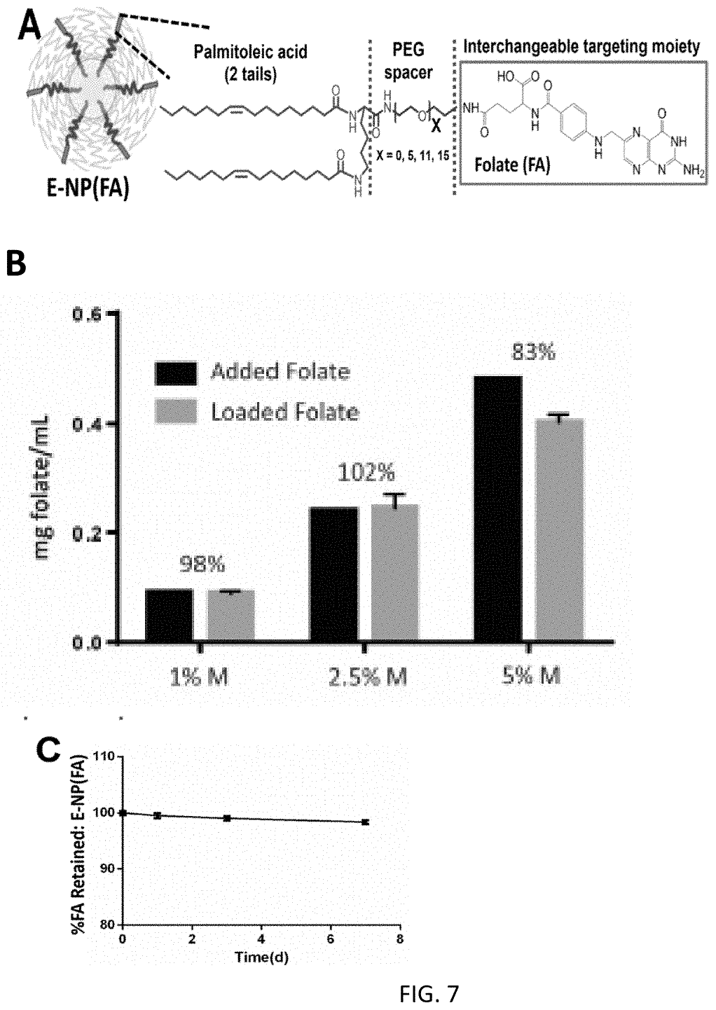

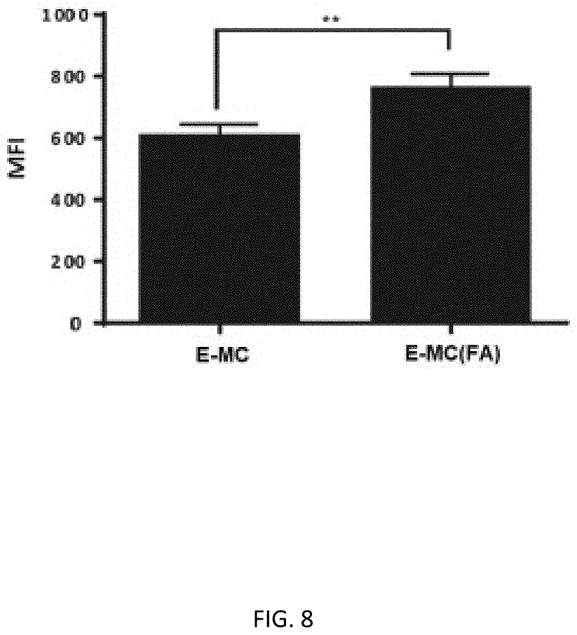

Having shown in vitro that (+) MiNP can transiently inhibit macropinocytosis while still allowing receptor-mediated endocytosis, we next sought to investigate whether (+) MiNP could enhance the uptake of chase nanoparticles targeting a specific cell receptor in vivo. The well-established B16F10 melanoma mouse model was chosen to compare the targeting of intratumoral folate receptors following IV and. SC routes of administration. B16F10 mouse melanoma cells have increased expression of folate receptors and folate decorated nanoparticles have been used by other groups to successfully target these cells.³⁴ We therefore synthesized a [folate]-[PEG linker]-[palmitoleic acid lipid anchor] (FA-PEG-PA) amphiphilic construct for stable incorporation into self-assembled PEG-b-PPS micelles (FIG. 1). Briefly, folate was attached to a PEG1k-amine spacer that was then linked to a palmitoleic acid tail using EDC (1-ethyl-3-[3-dimethylaminopropyl]-carbodiimide hydrochloride) chemistry. The resulting FA-PEG-PA construct was incorporated into micelles by shaking overnight in phosphate buffered saline, allowing the palmitoleic acid anchor to partition into the hydrophobic PPS core of the micelle. We have previously demonstrated that such lipid anchored constructs could be stably retained within self-assembled PEG-b-PPS nanoparticles for controlled surface display of targeting moieties, such as peptides.³⁵ The formation of folate displaying micelles (E-MC(FA)) at controllable molar ratios of PEG-b-PPS polymer to FA-PEG-PA construct was confirmed using UV-Vis spectroscopy (FIG. 7, ESI†). Additionally, the stability of the incorporation of the FA-PEG-PAlmitoleic acid construct and DiR dye was confirmed out to 7 days after initial formulation (FIGS. 5D and E, ESI†) An initial in vitro assessment by flow cytometry confirmed a significantly higher uptake of E-MC(FA) compared to E-MC following incubation with B16F10 melanoma cells for 1 h (FIG. 8, ESI†).

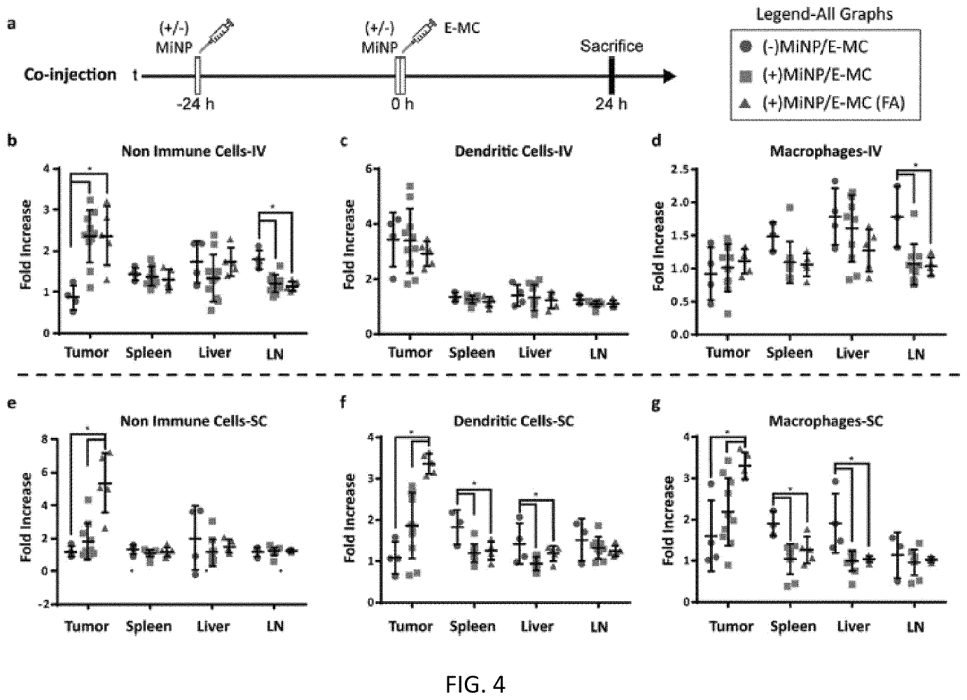

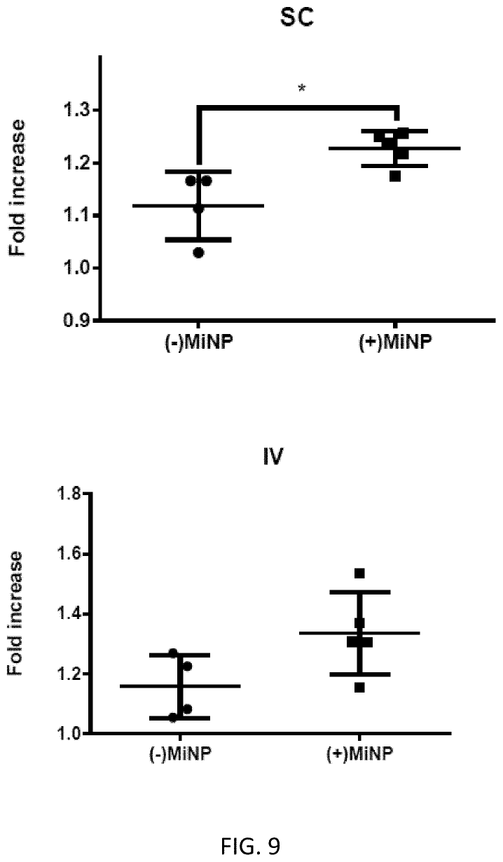

To investigate the enhancement in targeted delivery of MiNP co-administration, we compared the effect of our strategy on the uptake of folic-acid targeted E-MC(FA) vs. non-targeted E-MC. Mice were first inoculated with B16F10 melanoma cells, which were allowed to grow for approximately two weeks. Following an initial (+) MiNP or (−) MiNP injection at the −24 h timepoint, E-MC(FA) and E-MC were then coinjected with (+) MiNP or (−) MiNP using either SC or IV routes of administration (FIG. 4a). E-MC(FA) and E-MC uptake was assessed in the liver, lymph nodes, spleen and tumor using flow cytometry. DiR dye was used to identify both E-MC and E-MC(FA). IV injection of (+) MiNP increased E-MC uptake in tumor and decreased uptake in lymph node CD45-cells (non-immune) as well as lymph node macrophages (FIG. 4b-d). SC injection of (+) MiNP increased E-MC content in serum at 24 h while decreasing E-MC in liver and splenic DCs and macrophages (FIG. 9, ESI† and FIG. 4e-g). No differences were found in any of the tumor cell subsets using SC administration. These data indicate that IV injection of MiNPs could facilitate increased tumor targeting of an effector therapeutic, but that SC would not.

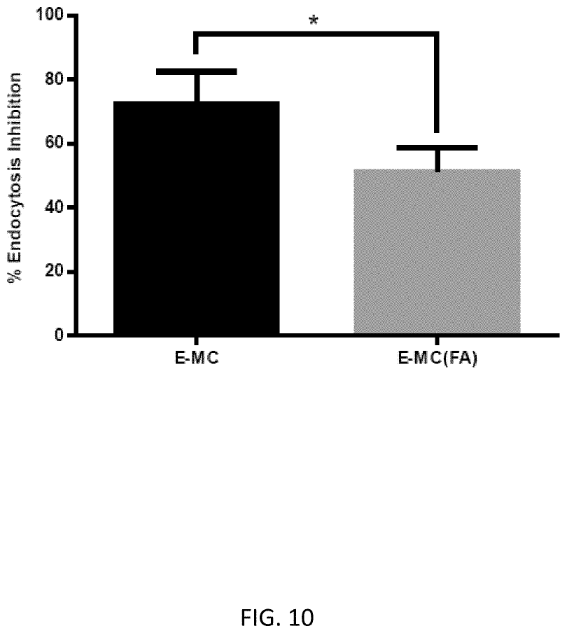

The administering of folate receptor targeted E-MC(FA) instead of E-MC had no significant effect when administered IV, but when administered SC, there was significantly increased uptake of E-MC(FA) in all tumor cell subsets (FIG. 4b-g). This increased tumor accumulation for targeted nanoparticles following only one of the routes of administration was unexpected, but may be explained by differences in E-MC clearance in MPS organs. Only the SC injection of (+) MiNP resulted in significant decreases in macrophage and dendritic cell uptake of E-MC in the spleen and liver, which would account for the additional E-MC(FA) available for accumulate in tumors. Our in vitro data demonstrated that MiNP inhibits macropinocytosis but does not strongly impact receptor mediated endocytosis (FIG. 2c, d and FIG. 10, ESI†), which we hypothesized would enhance the uptake of a receptor-targeted chase nanoparticle in vivo. Thus our co-administration of MiNP with E-MC possessing an additional folate receptor targeting element validated these in vitro results by significantly enhancing the accumulation of E-MC(FA) in B16F10 tumors up to 8-fold (FIG. 4e-g). This significant increase in uptake for E-MC(FA) versus E-MC was only observed within the solid tumors and in no other organs.

### CONCLUSION

Here, we demonstrate the nanoparticle biodistribution-altering effects of MiNP that encapsulate a small molecule inhibitor of macropinocytosis, LatA. We have characterized and evaluated these nanoparticles both in vitro and in vivo as key mediators in a co-administration strategy to increase the targeting efficacy of a second “effector” nanoparticle, E-MC. Clearance by the MPS remains a critical issue for many drug delivery applications beyond nanoparticles, suggesting a potentially broad range of applications for MiNP. For example, MPS organs are major sites of off-target accumulation for monoclonal antibodies' and decreasing this effect may allow enhanced efficacy with lower dosages and fewer side effects during cancer therapy. Strategies for blocking the clearance of therapeutic antibodies have long been under investigation,³⁷ yet inhibiting non-specific uptake via macropinocytosis remains underexplored. Recently, the depletion of subcapsular sinus macrophages via liposomes loaded with clodronate and other agents was employed to investigate the role of these cells during nanovaccination.³⁸ Results showed that removal of these cells prior to immunization enhanced delivery of the nanovaccine to lymph node follicles for improved humoral responses. Our work supports such strategies while additionally demonstrating that inhibition of MIPS cells can be performed in a reversible and nontoxic manner without killing phagocytes, many of which play critical downstream roles in the generation of an immune response. Furthermore, SC administration of MiNP increased the serum concentration of E-MC to levels similar to IV administration, potentially opening up new routes of administration and dosing regimens previously unavailable to many nanotherapeutics and controlled delivery strategies.

In a tumor model, we found that MiNP increase target tissue and cell accumulation through reduction of uptake by phagocytic cells such as macrophages and dendritic cells in the liver and spleen, which accounts for >90% of MPS cells. Our results validate LatA loaded PEG-b-PPS MiNP as a promising platform to improve the performance of other, paired effector nanoparticle therapeutic and diagnostic platforms. These proof-of-concept results justify the exploration of alternative MiNP formulations encapsulating inhibitors in addition to or in combination with LatA, as well as the investigation of MiNP as part of functional strategies employing effector nanoparticles loaded with diagnostic and/or therapeutic agents.

### REFERENCES

1 R. F. Pagels and R. K. Prud'homme, J. Controlled Release, 2015, 219, 519-535.
2 N. Desai, AAPS J, 2012, 14, 282-295.
3 A. Albanese, P. S. Tang and W. C. W. Chan, Annu. Rev. Biomed. Eng., 2012, 14, 1-16.
4 D. E. Owens and N. A. Peppas, Int. J. Pharm., 2006, 307, 93-102.
5 R. van Furth, Z. A. Cohn, J. G. Hirsch, J. H. Humphrey, W. G. Spector and H. L. Langevoort, Bull. W. H. O., 1972, 46, 845-852.
6 N. Bertrand and J. C. Leroux, J. Controlled Release, 2012, 161, 152-163.
7 S. Wilhelm, A. J. Tavares, Q. Dai, S. Ohta, J. Audet, H. F. Dvorak and W. C. W. Chan, Nat. Rev. Mater., 2016, 1, 1-12.
8 D. Dutta and J. G. Donaldson, Cell. Logist., 2012, 2, 203-208.
9 G. J. Doherty and H. T. McMahon, Annu. Rev. Biochem., 2009, 78, 857-902.
10 S. Xu, B. Z. Olenyuk, C. T. Okamoto and S. F. Hamm-Alvarez, Adv. Drug Delivery Rev., 2013, 65, 121-138.
11 N. G. Sosale, K. R. Spinier, C. Alvey and D. E. Discher, Curr. Opin. Immunol., 2015, 35, 107-112.
12 N. G. Sosale, T. Rouhiparkouhi, A. M. Bradshaw, R. Dimova, R. Lipowsky and D. E. Discher, Blood, 2015, 125, 542-552.
13 K. P. Garcia, K. Zarschler, L. Barbaro, J. A. Barreto, W. O'Malley, L. Spiccia, H. Stephan and B. Graham, Small, 2014, 10, 2516-2529.
14 S. Allen, O. Osorio, Y. G. Liu and E. Scott, J. Controlled Release, 2017, 262, 91-103.
15 S. D. Allen, Y. G. Liu, S. Bobbala, L. Cai, P. I. Hecker, R. Temel and E. A. Scott, Nano Res., 2018, 11, 5689-5703.
16 D. J. Dowling, E. A. Scott, A. Scheid, I. Bergelson, S. Joshi, C. Pietrasanta, S. Brightman, G. Sanchez-Schmitz, S. D. Van Haren, J. Ninkovic, D. Kats, C. Guiducci, A. de Titta, C. K. Bonner, S. Hirosue, M. A. Swartz, J. A. Hubbell and Levy, J. Allergy Clin. Immunol., 2017, 140, 1339-1350.
17 S. Yi, S. D. Allen, Y. G. Liu, B. Z. Ouyang, X. Li, P. Augsornworawat, E. B. Thorp and E. A. Scott, ACS Nano, 2016, 10, 11290-11303.
18 E. A. Scott, A. Stano, M. Gillard, A. C. Maio-Liu, M. A. Swartz and J. A. Hubbell, Biomaterials, 2012, 33, 6211-6219.
19 S. Bobbala, S. D. Allen and E. A. Scott, Nanoscale, 2018, 10, 5078-5088.
20 A. M. Bannunah, D. Vllasaliu, J. Lord and S. Stolnik, Mol. Pharmaceutics, 2014, 11, 4363-4373.
21 S. T. Reddy, A. J. van der Vlies, E. Simeoni, V. Angeli, G. J. Randolph, C. P. O'Neil, L. K. Lee, M. A. Swartz and J. A. Hubbell, Nat. Biotechnol., 2007, 25, 1159-1164.
22 V. Manolova, A. Flace, M. Bauer, K. Schwarz, P. Saudan and M. F. Bachmann, Eur. J. Immunol., 2008, 38, 1404-1413.
23 N. B. Karabin, S. Allen, H. K. Kwon, S. Bobbala, E. Firlar, T. Shokuhfar, K. R. Shull and E. A. Scott, Nat. Commun., 2018, 9, 624.
24 S. D. Allen, Y. G. Liu, T. Kim, S. Bobbala, S. Yi, X. Zhang, J. Choi and E. A. Scott, Biomater. Sci., 2019, 7, 657-668.
25 M. C. Louzao, I. R. Ares and E. Cagide, FEBS J., 2008, 275, 6067-6074.
26 W. M. Morton, K. R. Ayscough and P. J. McLaughlin, Nat. Cell Biol., 2000, 2, 376-378.
27 I. Spector, N. R. Shochet, D. Blasberger and Y. Kashman, Cell Motil. Cytoskeleton, 1989, 13, 127-144.
28 T. Stack, A. Vahabikashi, M. Johnson and E. Scott, J. Biomed. Mater. Res., Part A, 2018, 106, 1771-1779.
29 S. Cerritelli, C. P. O'Neil, D. Velluto, A. Fontana, M. Adrian, J. Dubochet and J. A. Hubbell, Langmuir, 2009, 25, 11328-11335.
30 H. Konishi, S. Kikuchi, T. Ochiai, H. Ikoma, T. Kubota, D. Ichikawa, H. Fujiwara, K. Okamoto, C. Sakakura, T. Sonoyama, Y. Kokuba, H. Sasaki, T. Matsui and E. Otsuji, Anticancer Res., 2009, 29, 2091-2097.
31 A. Doller, A. Badawi, T. Schmid, T. Brauβ, T. Pleli, D. M. zu Heringdorf, A. Piiper, J. Pfeilschifter and W. Eberhardt, Exp. Cell Res., 2015, 330, 66-80.
32 S. D. Allen, S. Bobbala, N. B. Karabin, M. Modak and E. A. Scott, ACS Appl. Mater. Interfaces, 2018, 10, 33857-33866.
33 D. R. Overby, E. H. Zhou, R. Vargas-Pinto, R. M. Pedrigi, R. Fuchshofer, S. T. Braakman, R. Gupta, K. M. Perkumas, J. M. Sherwood, A. Vahabikashi, Q. Dang, J. H. Kim, C. R. Ethier, W. D. Stamer, J. J. Fredberg and M. Johnson, Proc. Natl. Acad. Sci. U.S.A., 2014, 201410602, DOI: 10.1073/pnas.1410602111.
34 L. Cheng, H. Ma, M. Shao, Q. Fan, H. Lv, J. Peng, T. Hao, D. Li, C. Zhao and X. Zong, Mol. Med. Rep., 2017, 16, 1101-1108.
35 S. Yi, X. Zhang, M. H. Sangji, Y. Liu, S. D. Allen, B. Xiao, S. Bobbala, C. L. Braverman, L. Cai, P. I. Hecker, M. DeBerge, E. B. Thorp, R. E. Temel, S. I. Stupp and E. A. Scott, Adv. Funct. Mater., 2019, 29, 1904399.
36 C. Halma, M. R. Daha and L. A. van Es, Clin. Exp. Immunol., 1992, 89, 1-7.
37 S. B. Clarkson, R. P. Kimberly, J. E. Valinsky, M. D. Witmer, J. B. Bussel, R. L. Nachman and J. C. Unkeless, J. Exp. Med., 1986, 164, 474-489.
38 Y. N. Zhang, W. Poon, E. Sefton and W. C. W. Chan, ACS Nano, 2020, 14, 9478-9490.

### Example 2

Reference is made to the Supporting Information of the article “Enhancing subcutaneous injection and target tissue accumulation of nanoparticles via co-administration with macropinocytosis inhibitory nanoparticles (MiNP),” Nanoscale Horiz, 2021, 6, 393, the content of which is incorporated by reference in entirety.

The following example provides supporting information for the information disclosed in Example 1 as follows.

### Experimental Section

### Synthesis of PEG-b-PPS Copolymer

The self-assembling amphiphilic block copolymer PEG₄₅-b-PPS₂₃ was synthesized according to previously published protocols[1]. Briefly, PEG thioacetate was activated using sodium methoxide to initiate anionic ring opening polymerization of propylene sulfide. Following depletion of monomer, the propylene sulfide block was end capped with bromo benzene. Resulting polymer was double precipitated in methanol for purification and then evaluated using 1H NMR (CDCL3) and gel permeation chromatography (Thermo) using a waters styragel column with refractive index and UV-Vis detectors in a tetrahydrofuran (THF) mobile phase.

### Synthesis of Folate-PEG-Palmitoleic Acid Targeting Constructs

Folate targeting moieties were synthesized to selectively enhance the uptake of micelles by B16F10 melanoma cells. Folic acid-primary amine functionalized PEG (FA-PEG2k-NH2) was purchased from creative PEG works. Palmitoleic acid was covalently linked to FA-PEG2k-NH2 using an EDC reaction. Briefly, FA-PEG2k-NH2 was dissolved in DMF and EDC and palmitoleic acid were then added along with 4-dimethylamino pyradine as a catalyst. The reaction was allowed to run for 2 h and subsequently purified through double precipitation in cold diethyl ether. The resulting construct was characterized using 1H NMR (CDCL3) and gel permeation chromatography (Thermo) using a waters styragel column with refractive index and UV-Vis detectors in a THF mobile phase.

### Assembly and Loading of PEG-b-PPS Micelles

Micelles were assembled from the previously described PEG-b-PPS block copolymer using a cosolvent evaporation method[1]. PEG₄₅-b-PPS₂₃ block copolymer, latrunculin A in various concentrations (Cayman Chemical) and 3 uL DiI or DiR (Thermo)/10 mg polymer (if fluorescence was required) were dissolved in 1 mL dichloromethane (DCM). The resulting DCM solution was added dropwise to a scintillation vial containing 1 mL of sterile PBS stirred vigorously. DCM was allowed to evaporate over the course of 2 h. Micelles containing LatA were defined as (+) MiNP, while those without as (−) MiNP. Resulting micelles were then purified or set aside for addition of folate targeting moieties.

Folate targeted micelles (E-MC(FA)) were prepared through addition of 2.5% molar ratio of folate targeting moiety dissolved in DMSO. The micelle formulations were then placed on a shaker overnight to allow the palmitoleic acid tail to partition into the hydrophobic domain of the micelles. All formulations were then purified through gravity column chromatography using a Sephadex-LH20 column (Sigma) with a PBS mobile phase. Resulting micelles were then concentrated and further purified using a 10 k MWCO Zebaspin desalting column (Thermo).

Characterization of Micelle Formulations

Once micelles were purified, their properties were characterized using a variety of techniques. Dynamic light scattering (DLS) was used to evaluate the zeta potential, size distribution, and polydispersity index (PDI) of the micelles using a zetasizer nano (Malvern Instrument) with a 4 mW He—Ne 633 nm Laser at 1 mg/mL in PBS. PDI was determined using a two-parameter fit to the DLS correlation data. Micelle morphology was characterized by cryogenic transmission electron microscopy (Gatan) (CryoTEM) as previously described[2]. Small angle X-ray scattering (SAXS) experiments were performed at the DuPont-Northwestern-Dow Collaborative Access Team (DND-CAT) beamline at Argonne National Laboratory's Advanced Photon Source (Argonne, Ill., USA). PRIMUS 2.8.2 software was utilized to obtain a final scattering curve after solvent buffer subtraction and structural parameters of micelle samples were determined using a polymer micelle model fit in SaxsView. LatA encapsulation and efficiency was determined using a previously described HPLC method[3]. 100 μL of concentrated and purified micelles were frozen at −80° C. and subsequently lyophilized overnight. Resulting cakes were solubilized in methanol to extract LatA. After 1 h of extraction, the suspension was centrifuged to eliminate precipitated polymer. Supernatant was then analyzed for LatA content using HPLC. A static Methanol:Water (95:5) mobile phase was used on an Agilent C18 XDB-Eclipse column with absorption (235 nm) was used to evaluate LatA concentration. Standard curves were generated through serial dilution of LatA and subsequent lyophilization and methanol extraction of each standard.

Folate content of nanoparticles was determined using spectrophotometric analysis measuring absorption at 358 nm. Samples were analyzed in triplicate. Folate constructs in DMSO were serial diluted in PBS to obtain a standard curve. Blank micelles and PBS were used as baseline controls and their values were subtracted from FA-Micelles and FA-PEG-Palmitoleic acid standards respectively to create adjusted measurements to compensate for any interference from micelle polymer.

Stability of Micelle Formulations

0.5 mL each of DiI loaded Lat A Micelles ((+) MiNP), DiR loaded micelles (E-MC), and DiR and folic acid targeting construct loaded (E-MC(FA)) micelle formulations were placed in Slide-A-Lyzer MINI dialysis devices with 10 k MWCO filters (Thermo Scientific). Phosphate buffered saline (PBS) was used as a reservoir and 100 μL of each micelle formulation was removed after 24, 72, and 168 hours and stored at 4° C. until it could be analyzed. Lat A content and folic acid targeting construct content was determined by HPLC and spectrophotometry as previously described in this manuscript. DiI and DiR content was assessed by fluorescence intensity as measured by spectrophotometry (Spectramax M3) using excitation emission wavelengths of 549/565 nm and 750/780 nm respectively. Each sample was compared to a t=0 sample taken prior to dialysis, and percentage change from the t=0 sample for each analyte was reported.

Cell Culture

RAW 264.7 Macrophages were purchased from ATCC and cultured in T75 polystyrene tissue culture treated flasks (BD falcon) with DMEM (Life Technologies), 10% fetal bovine serum (FBS) (Gibco), and 1% penicillin/streptomycin (Life Technologies). Cells were passaged via mechanical cell scraping once they were 75-80% confluent.

B16F10 mouse melanoma cells were a generous gift from the laboratory of Dr. Bin Zhang at Northwestern University. B16F10 cells were cultured in T75 polystyrene tissue culture treated flasks (BD falcon) with DMEM (Life Technologies) plus 10% fetal bovine serum (FBS), and 1% penicillin/streptomycin (Life Technologies). Cells were passaged via trypsinization once 75-85% confluent.

Both cell types were stored in an incubator at 37° C. and 5% CO₂. Cells were passaged at a 1:4 ratio.

In Vitro Cell Uptake Assay

Cell uptake assays were performed with both RAW 264.7 macrophages and B16F10 cells. Cells were adhered to 24 well tissue culture treated polystyrene plates (Costar) and treated with 0.25 mg of micelles containing DiI or DiR for 1 h at 37° C. and 5% CO₂. Cells were subsequently washed and harvested via mechanical scraping or trypsinization. Cells were then transferred to flow tubes and spun down at 300× g for 5 min. Cells were subsequently stained with zombie aqua cell fixability dye (Biolegend) and fixed using IC fixation buffer (Biolegend). Flow cytometry was performed using FACS diva on a BD Fortessa flow cytometer (BD biosciences). Untreated cells of both types were used to subtract out background fluorescence, and flow data were analyzed using Cytobank software (Cytobank).

In Vitro Macrophage Cytotoxicity

RAW264.7 macrophages were adhered to 24 well tissue culture treated polystyrene plates (Costar) and treated with free LatA or (+) MiNP at concentrations ranging from 0 μM to 10 μM. Cells were incubated at 37° C. and 5% CO₂. After 4 h, cells were washed and harvested via mechanical cell scraping. Cells were then stained with zombie aqua cell viability dye and fixed using IC fixation buffer (Biolegend). Flow cytometry was performed using FACS diva software on a BD Fortessa flow cytometer (BD biosciences). Ethanol killed RAW 264.7 macrophages stained with zombie aqua were used as a positive control. Flow data were analyzed using Cytobank Software (Cytobank).

Cryogenic Transmission Electron Microscopy

Prior to applying sample, 200 mesh lacey copper grids were glow discharged. 4 μl of sample was applied to the grid. Blotting proceeded for 5 seconds with a blot offset of +0.5 mm, and was immediately plunged into liquid ethane using a FEI Vitrobot Mark III plunge freezing instrument. Grids were stored in liquid nitrogen. Samples were imaged using a JEOL JEM1230 LaB6 emission TEM operating at 100 keV. Plunge-frozen grids were held at −180° C. using a Gatan Cryo Transfer Holder. A Gatan Orius SC1000 CCD camera Model 831 was used to collect data. ImageJ software was used to process images.

B16F10 Melanoma Tumor Inoculation

B16F10 melanoma cells were expanded using tissue culture treated T25 and T75 flasks. Once at an appropriate number, cells were harvested via trypsinization. Cells were counted and diluted to 3.5 million cells/mL and aliquoted in 500 μL volumes into sterile containers. C57/BL6 mice at 6 weeks of age were injected subcutaneously into the right hindquarters with 200 of the cell suspension. Mice were then returned to their enclosure and tumor growth was monitored over the next 10 days. Treatments were performed on days 12-14 after tumor inoculation. All animal experimental procedures were performed according to protocols approved by the Northwestern Office for the Institutional Animal Care and Use Committee (animal protocol #IS00008841).

In Vivo Biodistribution Studies

All in vivo mouse studies followed one of two injection protocols. First, a pre-injection strategy was tested where (+/−) MiNP were administered twice at both 24 h and 4 h prior to injection of the chase effector nanoparticle. A simplified procedure was also evaluated wherein the (+/−) MiNP were administered alone just once to pre-condition the mouse, which was then followed 24 h later by a co-injected dose of a (+/−) MiNP and E-MC multi-nanoparticle formulation. Mice were then sacrificed at 24 h post (+/−) MiNP/E-MC injection. A dose of 100 of 7 μM (+) MiNP solution (approx. 0.55 mg/kg) was administered in each (+) MiNP injection. All injections were 100 μL in volume and the total injected amount of (+) MiNP or (−) MiNP and E-MC were equal.

Mice were sacrificed using cervical dislocation at 24 h and organs were harvested and placed in 24 well non tissue culture treated plates (Costar) in RPMI 1640 complete media (Gibco) on ice and were processed immediately. Once harvested, cells were isolated from each organ according to a different protocol (detailed below). Prior to staining, isolated cells were blocked with Cd16/32 blocking buffer for 15 min. Isolated cells were stained with CD11b (FITC), CD11c (BV421)), CD45(PerCp-Cy5.5), F4/80 (PE-Texas Red), CD19 (Pe-Cy7) anti mouse antibodies, and zombie aqua cell viability dye (Amcyan) for 45 min. All cell staining materials were obtained from Biolegend. E-MC were labeled with Vybrant DiR (Thermo Fisher).

Compensation controls were created from unstained mouse spleens. Cells were also isolated and stained from 3 mice that received no treatment. These mice were used as a baseline fluorescence control and used to calculate the relative fluorescence increase of E-MC in each of the different cell subsets (fold increase in FIG. 3 and FIG. 4). After staining, cells were washed and fixed with 1:1 cell staining buffer and cell fixation buffer and were processed using flow cytometry within 3 days of fixation. Flow cytometry data was analyzed using Cytobank software (Cytobank) and the gating strategy is shown in figure S1. Data are reported as fold increase of E-MC median fluorescence intensity over median fluorescence intensity of untreated mice. Significance was determined using students t-test within each cell subset.

Liver

Livers were incubated at 37° C. in 0.2 mg/mL Dnase (Sigma), 5000 U/mL Collagenase IV (Sigma) solution in RPMI media (Gibco) for 1 h with agitation every 10 min. After chemical digestion, livers were mechanically disrupted through a 40 micron cell strainer and the resulting suspensions were centrifuged at 50×g for 5 minutes. The supernatant was reclaimed and centrifuged at 50×g a second time and the supernatant was again reclaimed. The resulting suspension was then centrifuged at 400×g for 5 min to pellet remaining cells at which point the pellet was resuspended in red blood cell lysis buffer (Biolegend) and allowed to rest at 4° C. for 15 min. Then the suspension was diluted with PBS and centrifuged at 400×g to pellet the cells. Cells were then resuspended in cell staining buffer and transferred to flow tubes for staining.

Spleen

Spleens were mechanically disrupted and passed through a 40 micron cell strainer with RPMI media (Gibco). The resulting cell suspension was centrifuged at 400×g to pellet cells and then resuspended in RBC lysis buffer (Biolegend). This suspension was incubated for 15 min at 4° C., then diluted with PBS and centrifuged at 400×g for 5 min. The resulting pellet was then resuspended in cell staining buffer and transferred to flow tubes for staining.

Lymph Nodes

Draining lymph nodes were mechanically disrupted and passed through a 40 micron cell strainer with RPMI media (Gibco). The resulting cell suspension was centrifuged at 400×g to pellet cells. The pellet was then resuspended in cell staining buffer and transferred to flow tubes for staining.

B16F10 Tumor

Tumors were diced using biopsy punches and the resulting tissue sections were incubated at 37° C. in 0.2 mg/mL Dnase (Sigma), 5000 U/mL Collagenase IV (Sigma) solution in RPMI media (Gibco) for 1.5 h with agitation every 10 min. After chemical digestion, tumor tissue and collagenase solution were passed through a 40 micron cell strainer and the resulting cell suspensions were centrifuged at 50×g for 5 min. The supernatant was reclaimed and centrifuged at 50×g a second time and the supernatant was again reclaimed. The resulting cell suspension was then centrifuged at 400×g for 5 min to pellet remaining cells at which point the pellet was resuspended in red blood cell lysis buffer (Biolegend) and allowed to rest at 4° C. for 15 min. The resulting suspension was diluted with PBS and centrifuged at 400×g to pellet the cells. Cells were then resuspended in cell staining buffer and transferred to flow tubes for staining.

Serum

Whole blood was obtained from mice via retroorbital bleed. Approximately 100 of blood was obtained at each bleed. Immediately after whole blood was collected, it was allowed to coagulate at room temperature for 30 min. Samples were then centrifuged for 10 min at 1500 × g in a centrifuge at 4° C. Supernatant was isolated immediately after centrifugation and stored at 4° C. until analysis, which was no more than 4 h after serum isolation.

Statistical Analysis

All statistical analyses were performed using GraphPad Prism software (GraphPad Software; version 8.4.1). Each figure notes the specific type of statistical test utilized to determine statistical significance.

Author Contributions

All experiments were designed by TS and EAS. PEG-b-PPS polymer was synthesized by MF. Micelle formulations were made by TS. TEM and DLS was performed by MV. SAXS data was collected and analysed by SB. Animal work including injections, care, and endpoint assays were performed by YL, TS, and SB. Flow cytometry, antibody panel design and data analysis were done by TS. In vitro uptake and cytotoxicity assays were performed by TS. Figures were designed by TS, MV, ME, and EAS. Manuscript was written by TS, SB, and EAS.

### REFERENCES FOR EXAMPLE 2

[1] S. Cerritelli, C. P. O'Neil, D. Velluto, A. Fontana, M. Adrian, J. Dubochet, J. A. Hubbell, Langmuir 2009, 25, 11328.
[2] S. Allen, O. Osorio, Y. G. Liu, E. Scott, J. Control. Release Off. J. Control. Release Soc. 2017, 262, 91.
[3] T. Stack, A. Vahabikashi, M. Johnson, E. Scott, J. Biomed. Mater. Res. A 2018, 106, 1771.

Unless otherwise specified or indicated by context, the terms “a”, “an”, and “the” mean “one or more.” For example, “a molecule” should be interpreted to mean “one or more molecules.”

### Example 3

This Example demonstrates our novel “indirect” targeting strategy to enhance nanoparticle delivery to the TME by inhibiting non-specific clearance by the MPS. Our novel multi-nanoparticle strategy safely and temporarily inhibits macropinocytosis by MPS cells, while allowing receptor-mediated cellular targeting to continue unimpeded. In this method, macropinocytosis inhibitory nanoparticles (MiNP) are pre- or co-administered with “effector” nanoparticles (E-NP), the latter of which can perform either a diagnostic or therapeutic function. This “indirect” targeting results in modulation of off-target cells by MiNP enhances the circulation time, serum concentration, and thus targeting of E-NP without needing to directly modify the E-NP. This method is distinct from and complements known “active” and “passive” targeting strategies, which respectively employ targeting ligands or specific nanoparticle physicochemical properties for cell-selective uptake.

Our multi-nanoparticle formulations include scalable assembly and loading of bioactive biologics and small molecules into diverse nanostructure morphologies via flash nanoprecipitation. Flash nanoprecipitation for scalable assembly of diverse nanostructure morphologies employees confined impingement jets mixers' for the customizable self-assembly of poly(ethylene glycol)-b-poly(propylene sulfide) (PEG-b-PPS) copolymers into diverse nanostructures: solid-core spherical micelles (PEG₄₅-b-PPS₂₉, 20-40 nm diameter, MC), vesicular polymersomes (PEG₁₇-b-PPS₃₀, 100-150 nm diameter, PS), filamentous worm-like micelles (PEG₄₅-b-PPS₄₄, 10-30 nm diameter×1-2 micron length, FM) and bicontinuous nanospheres (PEG₁₇-b-PPS₇₅, 150-250 nm diameter, BCN). Each morphology has a unique organ and cellular biodistribution upon in vivo administration, which enables passive targeting strategies without the need for targeting ligands. Many of these structures are difficult to consistently form with other polymer systems, making PEG-b-PPS a uniquely versatile tool for nanoparticle fabrication.

Bicontinuous nanospheres for sustained intracellular release of therapeutics: We are the first to employ bicontinuous nanospheres (BCN) for in vivo delivery of therapeutic and diagnostic agents. These highly stable inverted colloids are characterized by a cubic lattice of aqueous channels that traverse a hydrophobic interior volume. BCN are the polymeric equivalent of lipid cubosomes, and their organized, lyotropic and interconnected internal architecture makes them exceptionally robust and stable assemblies. Relative to other nanostructures, like spherical micelles and vesicular polymersomes or liposomes, BCN are particularly amenable to high capacity dual loading of both hydrophobic and hydrophilic cargo, which respectively partition into the hydrophobic volume and aqueous channels. The internal aqueous channels allow BCN to slowly release payloads locally in a size-dependent manner, functioning as nanoscale analogs to more classical macroscale porous hydrogels. The enhanced stability of BCN allows them to retain and protect their payloads after endocytosis into cells, providing novel platforms for either triggered or sustained intracellular drug release¹⁸.

Engineer the nanostructure of MiNP for enhanced control over the duration of MPS inhibition.

The inverted lyotropic liquid crystal phases of BCN remain thermodynamically stable in excess water, and thus these self-assembled structures do not undergo micellar exchange due to equilibration with the surrounding aqueous environment. This has resulted in our novel application of BCNs as a slow-release intracellular delivery system amenable to the controlled release, low toxicity and scalable fabrication advantages of self-assembled nanoparticle systems. This Examples sets out to develop BCN as an alternative/complementary MiNP vehicle.

Assessment of BCN biodistribution. The inventors have previously demonstrated the drug loading efficiency, stability and biodistribution of BCN compared to more commonly used PS drug delivery vehicles¹⁶. BCN displayed higher loading efficiency as well as intra- and extracellular stability. Using IVIS, ICP-MS, and flow cytometry, we found BCN to primarily target MIPS cells within the spleen, with a rather even distribution between macrophages, dendritic cells and monocytes^{16,19}. The presence of BCN within the spleen gradually decreased over 7 days with no sign of toxicityl¹⁹.

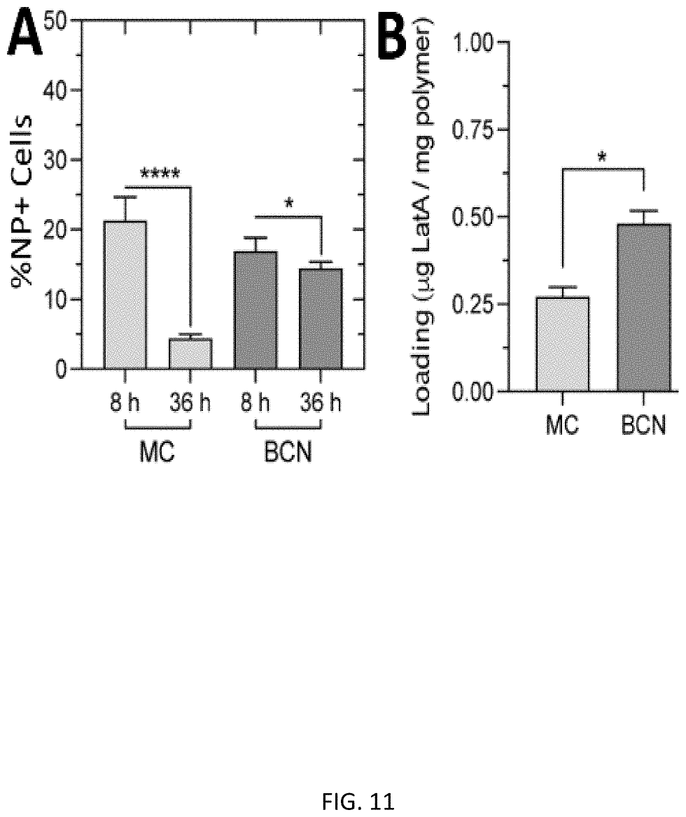

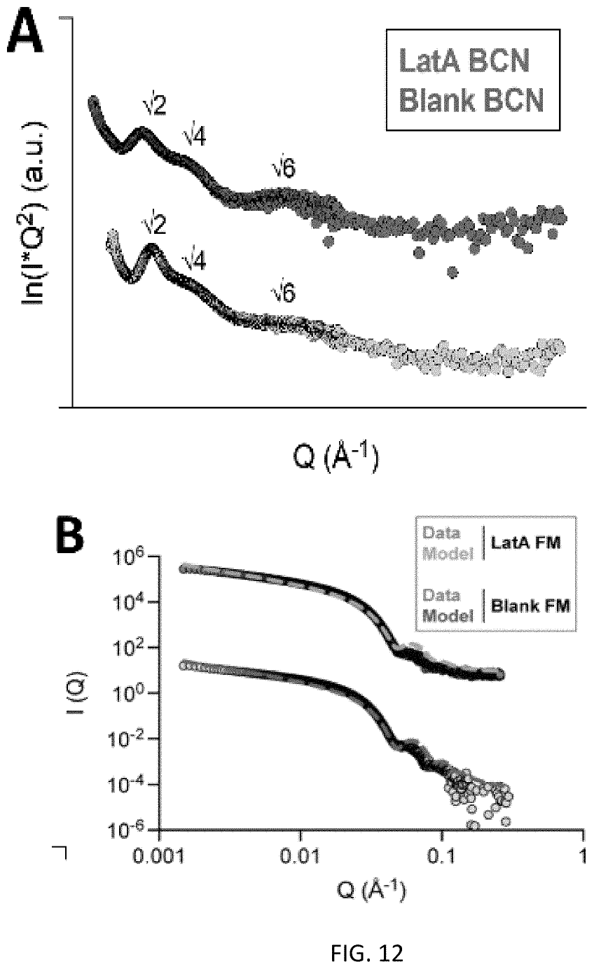

Here, we demonstrate fluorescently labeled BCN were shown to persist significantly longer than MC within cells (FIG. 11), verifying our published results of sustained intracellular drug delivery by BCN¹⁸. Furthermore, BCN showed an exceptional loading capacity for LatA, achieving more than double the loading capacity of MC (FIG. 11), suggesting that BCN may serve as an excellent depot for sustained intracellular delivery of LatA. Of note, endosomal BCN are capable of protecting cells from toxic effects of payloads at a high concentration that could otherwise result in cell death. SAXS demonstrated that BCNs loaded with LatA exhibit the expected 1 m3 m internal cubic organization (FIG. 12).

Comparison of BCN-based MiNP for toxicity and macropinocytosis inhibition. BCN will be validated first in vitro as a new MiNP vehicle via comparison with our previously validated micellar MiNP platform. While MC have a rapid intracellular release rate, the enhanced stability of the BCN structure is hypothesized to allow a sustained effect via slow release of LatA, as we have previously shown for other payloads. Established protocols used to characterize micellar MiNP will be employed for LatA-loaded BCN as described in Examples 1 and 2. As BCN persist longer in cells, toxicity will be assessed at additional timepoints: 24, 48, 72 & 108 h.

Optimization of administration schedule for slow release BCN-based MiNP. An injection schedule will be optimized for BCN as we have previously performed for micellar MiNP in Example 1 and 2. We will determine how long LatA-loaded BCN enhance the serum concentration of E-NP following a 48 h, 24 h and 12 h pre-injection in C57BL/6J mice. As with micellar MiNP, a second “booster” or co-injection will be performed to demonstrate enhancement of the inhibitory effect. Controls at different loaded concentrations of LatA per particle will be assessed to determine if BCN slow release and high LatA loading eliminate the need for booster strategies. In vivo toxicity studies in mice will be performed, as previously published⁴⁵, to verify the safety of extended intracellular release of LatA from BCN. RNA-seq will be performed, using our established protocols⁴⁸, to better characterize cells modulated by BCN-based MiNP and the impact of LatA on their in vivo gene expression and phenotype.

Enhanced delivery of E-NP to the TME using BCN-based MiNP pre-injection. Using the optimized injection schedule developed for enhanced E-NP circulation time, BCN-based MiNP will be investigated for improved delivery of E-NP(FA) to cells within the TME of B16F10 solid tumors. The concentration of LatA will be varied within the BCN (FIG. 11B) to modulate the duration of inhibition and potentially tune the uptake of E-NP within the TME. Target selectivity within the TME will be quantified by spectral flow cytometry.

Our preliminary data validating the MiNP/E-NP co-injection method employed the simplest form of MiNP (Example 1): small (20-40 nm diameter) fluorescently labeled methoxy functionalized micelles, which demonstrated extensive uptake in the liver and kidneys following s.c. injection. We anticipate significant differences in model E-NP targeting when using BCN-based MiNP. Due to the higher per cell dose of LatA delivered by each BCN nanoparticle (FIG. 11), slow intracellular release of LatA is anticipated to extend the inhibitory effect compared to micellar MiNP, resulting in a longer circulation time for E-NP and thus, a lower required LatA dosage overall. Furthermore, a slower release may eliminate the need for co-injection of MiNP with E-NP. Based upon their unique biodistribution, BCN should enhance inhibition in the spleen.

(2) Surface engineer MiNP for selective inhibition of MPS organs and improved control over indirect targeting of cells within the TME.

We will employ passive targeting strategies to rationally engineer MiNP for inhibition of critical MPS cell populations in the liver, kidney, blood, and spleen. The design parameters for these nanoparticles are outlined in Table 1, which are based upon the combined influence of morphology¹⁴ and surface chemistry² on adsorbed protein corona, which specifies targeting of distinct MPS organs and cells. The morphology will vary between MC (kidney preference), FM (blood preference), PS (liver preference) and BCN (spleen preference)¹⁹ which we have characterized extensively for preferential passive uptake by different phagocytic myeloid and innate immune cells in vivo. Surface chemistries utilized will be phosphate and methoxy, the former we have recently shown to possess unique biodistributions among macrophages and the latter being the most commonly employed nanoparticle surface chemistry².

Our indirect targeting methods¹¹ will be assessed for enhanced uptake of our model E-NP that has already been validated for targeting within the TME of B16F10 melanoma: DiR-labeled PEG-b-PPS MC presenting folate on their surfaces, E-NP(FA). E-NP(FA) will additionally be loaded with chemotherapeutics and immunomodulators to assess improved therapeutic efficacy in the treatment of melanoma. A key advantage of our MiNP strategy is the ability to employ simple s.c. injections, which we have already demonstrated to achieve systemic distribution of nanoparticles and tumor targeting on par with standard i.v. injections¹¹. This effect is likely due to inhibited clearance of nanoparticles at the site of injection and draining lymphatics, a process that we will further investigate in this study.

As nanoparticles are employed for both chemotherapy and immunotherapy, we have strategically selected chemotherapeutics and immunomodulators to assess enhanced efficacy during indirect targeting. This includes 3 small molecules that will be separately loaded within E-NP. Poorly water-soluble topoisomerase inhibitor camptothesin (CT) was selected as it has shown enhanced efficacy in mouse models of melanoma when delivered via nanoparticles⁵⁶. Furthermore, we have already optimized and employed PEG-b-PPS nanoparticles loaded with CT for controlled intracellular delivery to cancer cells¹⁸. Vemurafenib (PLX4032) is a BRAF inhibitor that has shown efficacy in the treatment of melanoma and will serve well to assess treatment in our BRAF (V600E)/PTEN melanoma mouse model⁵⁷. Regarding immunomodulators, imiquimod (IMQ) will be used as it is a TLR7/8 agonist with potent immunomodulatory effects on the TME that is approved for topical administration against melanoma⁵⁸⁻⁶¹, but has limited water solubility and a high potential to induce systemic immune responses and reactogenicity when not delivered in a controlled fashion in vivo. Due to its systemic effects on MIPS cell populations like macrophages and DCs, IMQ will assess both the therapeutic impact and lowered side effects resulting from inhibiting MPS cell populations during nanotherapy. All therapeutics will be administered at their published effective dosages. Quantification of loading will be performed using HPLC.

Surface chemistry-mediated modulation of adsorbed albumin folding state specifies nanoparticle clearance by distinct macrophage subsets: To investigate the influence of surface chemistry nanoparticle clearance by MPS cells, we synthesized and assembled hydroxyl-, methoxy- and phosphate-surface functionalized PS as model nanoparticle surface chemistries to probe physicochemical interactions at the nano/bio interface². Our initial studies revealed diverse murine macrophage subsets to be highly sensitive to PS surface chemistry in vivo, resulting in distinct nanoparticle biodistributions at both the organ and cell level. Follow-up investigations established that the neutral surfaces denatured albumin, decreasing its alpha-helix content, while the anionic phosphate surface (which is biomimetic of cell membranes) was stabilizing and instead increased the albumin alpha-helix content². Furthermore, we found that the denaturing surfaces are significantly recognized by macrophage class A1 scavenger receptors (SR-A1), whereas the stabilizing phosphate surfaces evaded SR-A1 altogether. We therefore established surface chemistry-mediated control of adsorbed albumin conformation as a mechanism for tuning macrophage recognition of soft PEG-b-PPS nanoparticles².

Assessment of solid tumor targeting and treatment. A small library of MiNP (Table 3) will be administered s.c. to C₅₇BL/6J mice bearing B16F10 and BRAF (V600E)/PTEN tumors using our published protocols¹¹. Briefly, B16F10 tumor will be established on their right flank (˜5×10⁵ cells/100 μl) and nanoparticles will be administered on either the same or opposite side. Since mutations in the serine-threonine protein kinase B-RAF (BRAFV600E) is present in over 50% of melanomas, BRAF (V600E)/PTEN mice present a clinically relevant model of de novo melanoma⁶³. For the inducible model, localized melanoma will be induced by application of 4-hydroxytamoxifen (4-HT) on day 0. Mice will receive MiNP/E-NP(FA) injections following the co-injection protocol (FIG. 7) twice per week or according to published regimen. Tumor size will be measured by caliper every 2 days for 30 days or until a maximum burden of 20 mm diameter is reached. Mice will then be sacrificed and tumor, organs (lymph nodes, liver, spleen, kidneys, and lung) and blood will be collected for analysis by IVIS and flow cytometry to assess uptake of fluorescent E-NP and modulation of the TME.

TABLE 3
MiNP library for selective inhibition of MPS cells.

| Target MPS Organ | Morphology | Surface Chemistry |
|---|---|---|
| Liver | PS | MeO |
| | | Phos |
| Kidney | MC | MeO |
| | | Phos |
| Spleen | BCN | MeO |
| | | Phos |
| Blood | FM | MeO |
| | | Phos |

Metastatic tumor targeting and treatment. A critical advantage of nanotherapy is the ability to target sites of metastasis that would otherwise be too small or disperse for detection and/or surgical removal. Both the B16F10 and models are known to metastasize to draining lymph nodes and lung⁶⁴. We will directly assess the impact of the MiNP/E-NP co-injection strategy on the targeting and treatment of sites of metastasis. B16F10 cells will be injected (˜5×10⁵ cells/100 μl) i.v. via the tail vein to allow metastasis of the lung. Mice will receive treatments (Table 3) on days 3, 7 and 11 with organs harvested on day 14 for characterization as previously described. To assess targeting, fluorescent nanoparticles will be employed for the final injections on day 11.

We anticipate significant differences in model E-NP therapeutic efficacy when co-injected with MiNP designed for selective inhibition of MPS cells in the lung, kidney and liver. Enhanced targeting of liver Kupffer cells by MiNP should further improve uptake in solid tumors at lower dosages than our original micellar MiNP design. MiNP focused on the kidney should decrease kidney toxicity of drug-loaded E-NP as assessed by blood chemistry (BUN and creatinine). MiNP targeting the liver should have enhanced direct effects on E-NP(FA) targeting of tumor cells in B16F10 compared to BRAF (V600E)/PTEN tumors, the latter of which do not have heightened folate receptor expression. Both tumor types should respond to IMQ-loaded E-NP immunotherapy, which will function mainly via myeloid cells in the TME and not be dependent on tumor cell targeting.

Indirect targeting for personalized cancer nanotherapy in a model of vascular inflammation comorbidity.

The impact of disease-specific biochemistry and inflammatory state on nanoparticle delivery is critically understudied. In particular, diseases involving chronic inflammation can significantly modulate the numbers and activation state of MPS cells²⁷. We have directly observed the impact of chronic vascular inflammation on nanoparticle biodistribution in mouse models of atherosclerosis, such as Ldlr^{-/-} mice that develop atherosclerotic plaques within the aortic arch when fed a high fat diet (HFD). In these mice, significantly heightened numbers of circulating inflammatory monocytes were found to rapidly clear nanoparticles from blood, drastically shifting the organ and cellular accumulation of nanotherapeutics¹⁴. Vascular inflammation is associated with the most common comorbidities in the United States, including obesity, metabolic syndrome, and diabetes in addition to atherosclerosis. We hypothesize that changes in MPS clearance resulting from these comorbidities will significantly impact the efficacy and dosing of nanotherapies during the treatment of cancer.

Here, we employ atherosclerosis as a model of comorbidity with melanoma and will rationally engineer a MiNP combination formulation to address disease specific changes in MIPS activity. Our preliminary data identified notable changes in the spleen, liver and blood, each of which we will be addressed via the incorporation of a specific MiNP.

To assess how a common comorbidity can change a nanoparticle's biodistribution within immune cells, the same concentration of methoxy functionalized DyLight 650-labeled PEG-b-PPS PS were injected i.v. (150 μL, 15 mg/mL) into both naïve C57BL/6J mice fed a standard chow diet and atherosclerotic Ldlr^{-/-} mice (C57BL/6J background) fed an HFD for 16 weeks¹⁴. The percentages of PS positive (PS⁺) macrophages were significantly lower in the spleen and liver of HFD-fed mice, while PS uptake by dendritic cells (DCs) remained unchanged in the spleen and significantly increased in the liver. Monocytosis is commonly observed during atherosclerosis wherein the bone marrow and spleen overproduce monocytes that enter the blood circulation and contribute to hypercholesterolemia²⁷. We hypothesized that increased levels of circulating monocytes in blood might contribute to the decreased presence of PS⁺ macrophages in the spleen and liver of the HFD-fed mice. Monocytosis doubled the blood monocyte concentration for HFD-fed mice with 30% of circulating blood monocytes being PS⁺. In comparison, less than 2% of circulating monocytes were PS⁺ in naïve mice, suggesting that both the quantity and inflammatory state of MPS cells play a role in shifting the nanoparticle biodistribution.

TME uptake of a combination MiNP formulation. Our preliminary data suggest that PS may passively target key MPS cells in the spleen, liver and blood contributing to nanoparticle clearance in atherosclerotic mice. We will therefore start with PS-based MiNP and assess enhancement in the formulation's efficacy by additionally incorporating BCN-based MiNP and FM-based MiNP to respectively improve inhibition in the spleen and blood (FIGS. 3 & 14)^{14,19}. After 16 weeks on HFD, B16F10 cells will be injected s.c. on their right flank (˜5×10⁵ cells/100 μl) of LdIr^{-/-} mice. Ldlr^{-/-} tumor-bearing mice fed a standard chow diet will serve as controls, since Ldlr^{-/-} require HFD to generate atheromas. The previously optimized co-injection schedule (FIG. 3A) will then be employed to assess E-NP(FA) circulation time and biodistribution, with the assessment of TME targeting by flow cytometry. MiNP formulations tested will include 1) (−) MiNP, 2) PS-based (+) MiNP, 3) PS/BCN-based MiNP, 4) PS/BCN/FM-based MiNP, 5) BCN/FM-based MiNP, 6) BCN-based MiNP 7) FM-based MiNP. For each formulation, the same total mass of polymer and the same dose of LatA will be employed. For combination formulations, the mass of polymer (1.5 mg) will be divided between the different morphologies.

A combination MiNP formulation in a comorbidity model of vascular inflammation and melanoma: The optimal MiNP formulation will be employed to assess the therapeutic treatment of LdIr^{-/-} with B16F10 melanoma and resulting sites of metastasis in the lung. The same Ldlr^{-/-} mouse model of atherosclerosis/melanoma comorbidity will be used. E-NP will be individually loaded with the chemotherapeutic and immunomodulatory drugs to evaluate changes in tumor size during treatment. We will assess enhanced E-NP(FA) targeting and treatment of B16F10 solid tumors as well as for sites of pseudometastasis induced by i.v. administration of B16F10 cells.

We anticipate PS-based MiNP alone to have a significant effect on tumor targeting, as PS were already shown to passively target up to 35% of circulating monocytes in atherosclerotic mice, a burden that resulted in significantly less uptake in the spleen and liver. The biodistribution of BCN in atherosclerotic mice is not currently known, but if they continue to target primarily the spleen, only an incremental enhancement is expected for the PS/BCN-based MiNP combined formulation. The most significant improvement over the PS-based MiNP is expected for the PS/FM-based MiNP, since FM were passively taken up by up to 80% of monocytes in standard mice (compared to <1% for PS). With a decreased clearance of E-NP in the blood, liver clearance may increase to the levels observed for normal mice, and thus maintaining a balance between PS and FM will allow both the liver and blood MPS cells to be inhibited. Further spreading the LatA between PS, BCN and FM may decrease inhibition of blood monocytes, resulting in lower efficacy for the PS/BCN/FM-based combined MiNP formulation. Balancing these three inhibitors will thus provide insight into how the MPS changes in a disease state of chronic inflammation.

Experimental assays to assess nanoparticles. Characterization of adsorbed protein composition over time: Protein adsorption characterization will use mouse and human plasma, since the various clotting components of the blood are often major protein corona constituents. The nanoparticle library will be incubated 1:1 with human/animal plasma samples at 37° C., 80 rpm. Sample aliquots will be retrieved at 2 h, 8 h, and 24 h. Multiple time points are included to assess the evolution of the formed protein coronas. Label-free proteomics: Label-free quantitation will be implemented as a modern proteomic technique to identify and quantify the relative abundance of protein corona constituents in 5 μg of adsorbed protein (submitted per sample). Samples will be desalted, digested with trypsin, and subjected to LC-MS/MS on a Q Exactive HF mass spectrometer (Thermo Fisher Scientific). Analysis will proceed from two technical replicates per sample. Proteins will be identified, and relative abundances determined using MaxQuant software. In vivo biodistribution assessment: We have designed labeling strategies for conjugation of combinations of fluorescent dyes and isotopic labels with minimal impact to the bio/nano interface, enabling the multimodal tracing of nanoparticles in vivo (FIGS. 3 & 11). Labeling will permit nanoparticle detection in live animals as well as in extracted blood and tissues by ICP-MS (parts per billion sensitivity via a synthetically incorporated ³³S tracer), in vivo imaging system (IVIS, DiR dye) and flow cytometry (DiR or DiD dye) with subsequent t-SNE analysis. Histology will be performed to validate results and assess local inflammation and toxicity. Toxicity assessment: Toxicity will be assessed over 4 weeks using weekly i.v. doses of nanoparticles from 0.1 to 200 mg/kg. Blood chemistry, weekly relative body weight and histology of organs (liver, spleen, kidneys, heart, lung and brain) will be assessed using our published protocols⁴⁵. Immunosuppression assessment: Total white blood cell levels will be monitored using weekly blood samples. Changes in immune cell composition in spleen, lymph nodes and blood will be monitored using comprehensive high parameter spectral flow cytometry as we previously performed and describe below⁴⁸.

Treatment assessment of organs and tumors via high parameter spectral flow cytometry with tSNE analysis. The following tissues will be collected and processed for flow cytometry: blood, kidneys, liver, lung, lymph nodes (collected separately) including axial, brachial, inguinal, mesenteric, pancreaticoduodenal, spleen and thymus. Diverse MIPS cell populations, including monocytes, macrophages, dendritic cells and endothelial cells will be assessed by including the following markers: B220, CD3, CD4, CD8, CD11b, CD11c, CD19, CD25, CD28, CD40, CD40L, CD45, CD68, CD69, CD80, CD86, F4/80, FoxP3, IDO, IL-2, IL-10, Ly-6C, Ly-6G, NK1.1, MHC I, MHC II, PD-1, CD31, CD34, ICAM-1, Tie-2/Tek, VCAM-1, VEGF, VEGFR-3, LYVE-1 and VE-cadherin. tSNE analysis will be performed using FlowJo's DownSample plugin to randomly select an equal number of events from each cell population (for example, CD45+, CD3+, CD19+, CD11b+, CD11c+, etc.) of every sample. DownSample will both normalize the contribution of each mouse replicate and reduce the computational burden⁷⁰.

### REFERENCES FOR EXAMPLE 3

### References

1. Karabin N B, Vincent M P, Allen S D, Bobbala S, Frey M A, Yi S, Yang Y, Scott E A. The Combination of Morphology and Surface Chemistry Defines the Biological Identity of Nanocarriers in Human Blood. bioRxiv. 2020. doi: 10.1101/2020.09.02.280404.
2. Vincent M P, Bobbala S, Karabin N B, Frey M, Liu Y, Navidzadeh J O, Stack T, Scott E A. Surface chemistry-mediated modulation of adsorbed albumin folding state specifies nanocarrier clearance by distinct macrophage subsets. Nat Commun. 2021; 12(1): 648. Epub 2021/01/30. doi: 10.1038/s41467-020-20886-7. PubMed PMID: 33510170; PMCID: PMC7844416.
3. Nagaraj R, Stack T, Yi S, Mathew B, Shull K R, Scott E A, Mathew M T, Bijukumar D R. High Density Display of an Anti-Angiogenic Peptide on Micelle Surfaces Enhances Their Inhibition of αvfβ3 Integrin-Mediated Neovascularization In Vitro. Nanomaterials (Basel). 2020; 10 (3). Epub 2020/04/03. doi: 10.3390/nano10030581. PubMed PMID: 32235802; PMCID: PMC7153711.
4. Stack T, Vincent M, Vahabikashi A, Li G, Perkumas K M, Stamer W D, Johnson M, Scott E A. Targeted Delivery of Cell Softening Micelles to Schlemm's Canal Endothelial Cells for Treatment of Glaucoma. Small. 2020. doi: 10.1002/smll.202004205.
5. Yi S, Zhang X, Sangji M H, Liu Y, Allen S D, Xiao B, Bobbala S, Braverman C L, Cai L, Hecker P I, DeBerge M, Thorp E B, Temel R E, Stupp S I, Scott E A. Surface Engineered Polymersomes for Enhanced Modulation of Dendritic Cells During Cardiovascular Immunotherapy. Advanced Functional Materials. 2019; 29(42): 1904399. doi: 10.1002/adfm.201904399.
6. Scott E A, Karabin N B, Augsornworawat P. Overcoming Immune Dysregulation with Immunoengineered Nanobiomaterials. Annu Rev Biomed Eng. 2017; 19: 57-84. Epub 2017/02/23. doi: 10.1146/annurev-bioeng-071516-044603. PubMed PMID: 28226216.
7. Wilhelm S, Tavares A J, Dai Q, Ohta S, Audet J, Dvorak H F, Chan W C W. Analysis of nanoparticle delivery to tumours. Nat Rev Mater. 2016; 1(5): 16014. doi: 10.1038/natrevmats.2016.14.
8. Torrice M. Does Nanomedicine Have a Delivery Problem? ACS Cent Sci. 2016; 2(7): 434-7. Epub 2016/08/10. doi: 10.1021/acscentsci.6b00190. PubMed PMID: 27504489; PMCID: PMC4965851.
9. Zhang H, Yang Y, Steinbrecher U P. Structural requirements for the binding of modified proteins to the scavenger receptor of macrophages. J Biol Chem. 1993; 268(8): 5535-42. Epub 1993/03/15. PubMed PMID: 8383674.
10. Patel P C, Giljohann D A, Daniel W L, Zheng D, Prigodich A E, Mirkin C A. Scavenger receptors mediate cellular uptake of polyvalent oligonucleotide-functionalized gold nanoparticles. Bioconjug Chem. 2010; 21(12): 2250-6. Epub 2010/11/13. doi: 10.1021/bc1002423. PubMed PMID: 21070003; PMCID: PMC3241523.
11. Stack T, Liu Y, Frey M, Bobbala S, Vincent M, Scott E. Enhancing subcutaneous injection and target tissue accumulation of nanoparticles via co-administration with macropinocytosis inhibitory nanoparticles (MiNP). Nanoscale Horiz. 2021. Epub 2021/04/23. doi: 10.1039/d0nh00679c. PubMed PMID: 33884386.
12. Stack T, Vincent M, Vahabikashi A, Li G, Perkumas K M, Stamer W D, Johnson M, Scott E. Targeted Delivery of Cell Softening Micelles to Schlemm's Canal Endothelial Cells for Treatment of Glaucoma. Small. 2020; 16 (43): e2004205. Epub 2020/10/06. doi: 10.1002/smll.202004205. PubMed PMID: 33015961; PMCID: PMC7647937.
13. Allen S, Vincent M, Scott E. Rapid, Scalable Assembly and Loading of Bioactive Proteins and Immunostimulants into Diverse Synthetic Nanocarriers Via Flash Nanoprecipitation. J Vis Exp. 2018 (138). Epub 2018/08/28. doi: 10.3791/57793. PubMed PMID: 30148497; PMCID: PMC6126708.
14. Yi S, Allen S D, Liu Y G, Ouyang B Z, Li X, Augsornworawat P, Thorp E B, Scott E A. Tailoring Nanostructure Morphology for Enhanced Targeting of Dendritic Cells in Atherosclerosis. ACS Nano. 2016; 10(12): 11290-303. doi: 10.1021/acsnano.6b06451. PubMed PMID: 27935698; PMCID: PMC5418862.
15. Allen S, Osorio O, Liu Y G, Scott E. Facile assembly and loading of theranostic polymersomes via multi-impingement flash nanoprecipitation. J Control Release. 2017; 262: 91-103. Epub 2017/07/25. doi: 10.1016/j.jconre1.2017.07.026. PubMed PMID: 28736263; PMCID: PMC5603398.
16. Allen S D, Bobbala S, Karabin N B, Modak M, Scott E A. Benchmarking Bicontinuous Nanospheres against Polymersomes for in Vivo Biodistribution and Dual Intracellular Delivery of Lipophilic and Water-Soluble Payloads. ACS Applied Materials & Interfaces. 2018; 10(40): 33857-66. doi: 10.1021/acsami.8b09906.
17. Bobbala S, Allen S D, Scott E A. Flash nanoprecipitation permits versatile assembly and loading of polymeric bicontinuous cubic nanospheres. Nanoscale. 2018; 10(11): 5078-88. Epub 2017/12/20. doi: 10.1039/c7nr06779h. PubMed PMID: 29255814.
18. Bobbala S, Allen S D, Yi S, Vincent M, Frey M, Karabin N B, Scott E A. Employing bicontinuous-to-micellar transitions in nanostructure morphology for on-demand photo-oxidation responsive cytosolic delivery and off-on cytotoxicity. Nanoscale. 2020; 12(9): 5332-40. doi: 10.1039/C₉NR10921H.
19. Modak M, Bobbala S, Lescott C, Liu Y G, Nandwana V, Dravid V P, Scott E A. Magnetic Nanostructure-Loaded Bicontinuous Nanospheres Support Multicargo Intracellular Delivery and Oxidation-Responsive Morphological Transitions. ACS Appl Mater Interfaces. 2020; 12(50): 55584-95. Epub 2020/12/02. doi: 10.1021/acsami.0c15920. PubMed PMID: 33259182.
20. Frey M, Bobbala S, Karabin N, Scott E. Influences of nanocarrier morphology on therapeutic immunomodulation. Nanomedicine (Lond). 2018; 13(14): 1795-811. Epub 2018/08/08. doi: 10.2217/nnm-2018-0052. PubMed PMID: 30084296; PMCID: PMC7270889.
21. Allen S, Liu Y G, Scott E. Engineering nanomaterials to address cell-mediated inflammation in atherosclerosis. Regen Eng Transl Med. 2016; 2(1): 37-50. Epub 2016/05/03. doi: 10.1007/s40883-016-0012-9. PubMed PMID: 27135051; PMCID: PMC4849406.
22. Wilhelm S, Tavares A J, Dai Q, Ohta S, Audet J, Dvorak H F, Chan W C W. Analysis of nanoparticle delivery to tumours. Nature Reviews Materials. 2016; 1: 16014. doi: 10.1038/natrevmats.2016.14 https://www.nature.com/articles/natrevmats201614#supplementary-information.
23. Albanese A, Tang P S, Chan W C. The effect of nanoparticle size, shape, and surface chemistry on biological systems. Annual review of biomedical engineering. 2012; 14: 1-16. doi: 10.1146/annurev-bioeng-071811-150124. PubMed PMID: 22524388.
24. Barenholz Y. Doxil®—the first FDA-approved nano-drug: lessons learned. J Control Release. 2012; 160(2): 117-34. Epub 2012/04/10. doi: 10.1016/j.jconre1.2012.03.020. PubMed PMID: 22484195.
25. Park K. Facing the truth about nanotechnology in drug delivery. ACS Nano. 2013; 7(9): 7442-7. Epub 2014/02/05. doi: 10.1021/nn404501g. PubMed PMID: 24490875; PMCID: PMC3914010.
26. Tacconi C, Commerford C D, Dieterich L C, Schwager S, He Y, Ikenberg K, Friebel E, Becher B, Tugues S, Detmar M. CD169 (+) lymph node macrophages have protective functions in mouse breast cancer metastasis. Cell Rep. 2021; 35(2): 108993. Epub 2021/04/15. doi: 10.1016/j.celrep.2021.108993. PubMed PMID: 33852863.
27. Swirski F K, Nahrendorf M. Leukocyte Behavior in Atherosclerosis,
Myocardial Infarction, and Heart Failure. Science. 2013; 339(6116): 161-6. doi: 10.1126/science.1230719.
28. Flynn M C, Pernes G, Lee M K S, Nagareddy P R, Murphy A J. Monocytes,
Macrophages, and Metabolic Disease in Atherosclerosis. Front Pharmacol. 2019; 10: 666. Epub 2019/06/30. doi: 10.3389/fphar.2019.00666. PubMed PMID: 31249530; PMCID: PMC6584106.
29. Hensel M, Gradel L, Kutz A, Haubitz S, Huber A, Mueller B, Schuetz P, Hugle T. Peripheral monocytosis as a predictive factor for adverse outcome in the emergency department: Survey based on a register study. Medicine (Baltimore). 2017; 96 (28): e7404. Epub 2017/07/13. doi: 10.1097/MD.0000000000007404. PubMed PMID: 28700476; PMCID: PMC5515748.
30. Barrett T J, Murphy A J, Goldberg I J, Fisher E A. Diabetes-mediated myelopoiesis and the relationship to cardiovascular risk. Ann N Y Acad Sci. 2017; 1402(1): 31-42. Epub 2017/09/20. doi: 10.1111/nyas.13462. PubMed PMID: 28926114; PMCID: PMC5659728.
31. Clemons T D, Singh R, Sorolla A, Chaudhari N, Hubbard A, Iyer K S. Distinction Between Active and Passive Targeting of Nanoparticles Dictate Their Overall Therapeutic Efficacy. Langmuir. 2018; 34(50): 15343-9. Epub 2018/11/18. doi: 10.1021/acs.langmuir.8b02946. PubMed PMID: 30441895.
32. Stack T, Vahabikashi A, Johnson M, Scott E. Modulation of Schlemm's canal endothelial cell stiffness via latrunculin loaded block copolymer micelles. J Biomed Mater Res A. 2018; 106(7): 1771-9. Epub 2018/02/23. doi: 10.1002/jbm.a.36376. PubMed PMID: 29468812; PMCID: PMC5980717.
33. Du F, Bobbala S, Yi S, Scott E A. Sequential intracellular release of water-soluble cargos from Shell-crosslinked polymersomes. J Control Release. 2018. Epub 2018/03/31. doi: 10.1016/j.jconrel.2018.03.027. PubMed PMID: 29601932.
34. Han J, Zhu Z, Qian H, Wohl A R, Beaman C J, Hoye T R, Macosko C W. A simple confined impingement jets mixer for flash nanoprecipitation. J Pharm Sci. 2012; 101(10): 4018-23. doi: 10.1002/jps.23259. PubMed PMID: 22777753.
35. Napoli A, Valentini M, Tirelli N, Muller M, Hubbell J A. Oxidation-responsive polymeric vesicles. Nat Mater. 2004; 3(3): 183-9. doi: 10.1038/nmat1081. PubMed PMID: 14991021.
36. Nicol E, Nicolai T, Durand D. Dynamics of poly(propylene sulfide) studied by dynamic mechanical measurements and dielectric spectroscopy. Macromolecules. 1999; 32(22): 7530-6. doi: DOI 10.1021/ma990853f. PubMed PMID: WOS:000083557200026.
37. Cerritelli S, Fontana A, Velluto D, Adrian M, Dubochet J, De Maria P, Hubbell J A. Thermodynamic and kinetic effects in the aggregation behavior of a poly(ethylene glycol-b-propylene sulfide-b-ethylene glycol) ABA triblock copolymer. Macromolecules. 2005; 38(18): 7845-51. doi: 10.1021/ma051176u. PubMed PMID: WOS:000231612000038.
38. Dowling D J, Scott E A, Scheid A, Bergelson I, Joshi S, Pietrasanta C, Brightman S, Sanchez-Schmitz G, Van Haren S D, Ninkovic J, Kats D, Guiducci C, de Titta A, Bonner D K, Hirosue S, Swartz M A, Hubbell J A, Levy O. Toll-like receptor 8 agonist nanoparticles mimic immunomodulating effects of the live BCG vaccine and enhance neonatal innate and adaptive immune responses. J Allergy Clin Immunol. 2017; 140(5): 1339-50. Epub 2017/03/28. doi: 10.1016/j.jaci.2016.12.985. PubMed PMID: 28343701; PMCID: PMC5667586.
39. Scott E A, Stano A, Gillard M, Maio-Liu A C, Swartz M A, Hubbell J A. Dendritic cell activation and T cell priming with adjuvant- and antigen-loaded oxidation-sensitive polymersomes. Biomaterials. 2012; 33(26): 6211-9. doi: 10.1016/j.biomaterials.2012.04.060. PubMed PMID: 22658634.
40. Stano A, Scott E A, Dane K Y, Swartz M A, Hubbell J A. Tunable T cell immunity towards a protein antigen using polymersomes vs. solid-core nanoparticles. Biomaterials. 2013; 34(17): 4339-46. doi: 10.1016/j.biomaterials.2013.02.024. PubMed PMID: 23478034.
41. Vasdekis A E, Scott E A, O'Neil C P, Psaltis D, Hubbell J A. Precision intracellular delivery based on optofluidic polymersome rupture. ACS nano. 2012; 6(9): 7850-7. doi: 10.1021/nn302122h. PubMed PMID: 22900579.
42. Napoli A, Valentini M, Tirelli N, Müller M, Hubbell J A. Oxidation-responsive polymeric vesicles. Nature Materials. 2004; 3: 183. doi: 10.1038/nmat1081 https://www.nature.com/articles/nmat1081#supplementary-information.
43. Olsen A W, Brandt L, Agger E M, van Pinxteren L A, Andersen P. The influence of remaining live BCG organisms in vaccinated mice on the maintenance of immunity to tuberculosis. Scand J Immunol. 2004; 60(3): 273-7. Epub 2004/08/24. doi: 10.1111/j.0300-9475.2004.01471.x. PubMed PMID: 15320884.
44. Napoli A, Boerakker M J, Tirelli N, Nolte R J M, Sommerdijk N A J M, Hubbell J A. Glucose-oxidase based self-destructing polymeric vesicles. Langmuir. 2004; 20(9): 3487-91. doi: Doi 10.1021/La0357054. PubMed PMID: WOS:000221022400001.
45. Allen S D, Liu Y-G, Bobbala S, Cai L, Hecker P I, Temel R, Scott E A. Polymersomes scalably fabricated via flash nanoprecipitation are non-toxic in nonhuman primates and associate with leukocytes in the spleen and kidney following intravenous administration. Nano Research. 2018. doi: 10.1007/s12274-018-2069-x.
46. Allen S D, Bobbala S, Karabin N B, Scott E A. On the advancement of polymeric bicontinuous nanospheres toward biomedical applications. Nanoscale Horiz. 2019; 4(2): 258-72. doi: 10.1039/c8nh00300a. PubMed PMID: WOS:000461558200002.
47. Larsson K. Cubic Lipid-Water Phases—Structures and Biomembrane Aspects. J Phys Chem-Us. 1989; 93(21): 7304-14. doi: DOI 10.1021/j100358a010. PubMed PMID: WOS:A1989AX04900010.
48. Burke J A, Zhang X, Bobbala S, Frey M A, Furentes C B, Haddad H F, Allen S D, Richardson R, Ameer G A, Scott E A. Subcutaneous nanotherapy repurposes the immunosuppressive mechanism of rapamycin to enhance allogeneic islet graft viability. Nature Nanotechnology. 2021. Epub In Revision. doi: 10.21203/rs.3.rs-92015/v1.
49. Dowling D J, Scott E A, Scheid A, Bergelson I, Joshi S, Pietrasanta C, Brightman S, Sanchez-Schmitz G, Van Haren S D, Ninkovic J, Kats D, Guiducci C, de Titta A, Bonner D K, Hirosue S, Swartz M A, Hubbell J A, Levy O. Toll-like receptor 8 agonist nanoparticles mimic immunomodulating effects of the live BCG vaccine and enhance neonatal innate and adaptive immune responses. J Allergy Clin Immunol. 2017. Epub 2017/03/28. doi: 10.1016/j.jaci.2016.12.985. PubMed PMID: 28343701.
50. Allen S D, Liu Y G, Kim T, Bobbala S, Yi S, Zhang X, Choi J, Scott E A. Celastrol-loaded PEG-b-PPS nanocarriers as an anti-inflammatory treatment for atherosclerosis. Biomaterials Science. 2019; 7(2): 657-68. doi: 10.1039/C8BM01224E.
51. Qiu H, Caffrey M. The phase diagram of the monoolein/water system: metastability and equilibrium aspects. Biomaterials. 2000; 21(3): 223-34. Epub 2000/01/26. doi: 10.1016/s0142-9612(99)00126-x. PubMed PMID: 10646938.
52. Bartneck M, Warzecha K T, Tacke F. Therapeutic targeting of liver inflammation and fibrosis by nanomedicine. Hepatobiliary Surg Nutr. 2014; 3(6): 364-76. Epub 2015/01/09. doi: 10.3978/j.issn.2304-3881.2014.11.02. PubMed PMID: 25568860; PMCID: PMC4273112.
53. Guo Y, Liu X, Sun X, Zhang Q, Gong T, Zhang Z. Mannosylated lipid nano-emulsions loaded with lycorine-oleic acid ionic complex for tumor cell-specific delivery. Theranostics. 2012; 2(11): 1104-14. Epub 2012/12/12. doi: 10.7150/thno.4525. PubMed PMID: 23227126; PMCID: PMC3516834.
54. Leroueil P R, Hong S, Mecke A, Baker J R, Jr., Orr B G, Banaszak Holl M M. Nanoparticle interaction with biological membranes: does nanotechnology present a Janus face? Acc Chem Res. 2007; 40(5): 335-42. Epub 2007/05/04. doi: 10.1021/ar600012y. PubMed PMID: 17474708; PMCID: PMC2551762.
55. Arvizo R R, Miranda O R, Moyano D F, Walden C A, Gin K, Bhattacharya R, Robertson J D, Rotello V M, Reid J M, Mukherjee P. Modulating pharmacokinetics, tumor uptake and biodistribution by engineered nanoparticles. PLoS One. 2011; 6 (9):e24374. Epub 2011/09/21. doi: 10.1371/journal.pone.0024374. PubMed PMID: 21931696; PMCID: PMC3172229.
56. Liu X P, Zhou S T, Li X Y, Chen X C, Zhao X, Qian Z Y, Zhou L N, Li Z Y, Wang Y M, Zhong Q, Yi T, Li Z Y, He X, Wei Y Q. Anti-tumor activity of N-trimethyl chitosan-encapsulated camptothecin in a mouse melanoma model. J Exp Clin Cancer Res. 2010; 29: 76. Epub 2010/06/23. doi: 10.1186/1756-9966-29-76. PubMed PMID: 20565783; PMCID: PMC2896352.
57. Hooijkaas A I, Gadiot J, van der Valk M, Mooi W J, Blank C U. Targeting BRAFV600E in an inducible murine model of melanoma. Am J Pathol. 2012; 181(3): 785-94. Epub 2012/07/17. doi: 10.1016/j.ajpath.2012.06.002. PubMed PMID: 22796458.
58. Wolf I H, Smolle J, Binder B, Cerroni L, Richtig E, Kerl H. Topical imiquimod in the treatment of metastatic melanoma to skin. Archives of dermatology. 2003; 139(3): 273-6. PubMed PMID: 12622616.
59. Naylor M F, Crowson N, Kuwahara R, Teague K, Garcia C, Mackinnis C, Hague R, Odom C, Jankey C, Cornelison R L. Treatment of lentigo maligna with topical imiquimod. The British journal of dermatology. 2003; 149 Suppl 66: 66-70. PubMed PMID: 14616356.
60. Furudate S, Fujimura T, Kambayashi Y, Kakizaki A, Hidaka T, Aiba S. Immunomodulatory Effect of Imiquimod Through CCL22 Produced by Tumor-associated Macrophages in B16F10 Melanomas. Anticancer Res. 2017; 37(7): 3461-71. Epub 2017/07/03. doi: 10.21873/anticanres.11714. PubMed PMID: 28668835.
61. Aspord C, Tramcourt L, Leloup C, Molens J P, Leccia M T, Charles J, Plumas J. Imiquimod inhibits melanoma development by promoting pDC cytotoxic functions and impeding tumor vascularization. J Invest Dermatol. 2014; 134(10): 2551-61. Epub 2014/04/23. doi: 10.1038/jid.2014.194. PubMed PMID: 24751730.
62. Karabin N B, Allen S, Kwon H K, Bobbala S, Firlar E, Shokuhfar T, Shull K R, Scott E A. Sustained micellar delivery via inducible transitions in nanostructure morphology. Nat Commun. 2018; 9(1): 624. Epub 2018/02/13. doi: 10.1038/s41467-018-03001-9. PubMed PMID: 29434200; PMCID: PMC5809489.
63. Knight D A, Ngiow S F, Li M, Parmenter T, Mok S, Cass A, Haynes N M, Kinross K, Yagita H, Koya R C, Graeber T G, Ribas A, McArthur G A, Smyth M J. Host immunity contributes to the anti-melanoma activity of BRAF inhibitors. J Clin Invest. 2013; 123(3): 1371-81. doi: 10.1172/JCI66236. PubMed PMID: 23454771; PMCID: PMC3582139.
64. Dankort D, Curley D P, Cartlidge R A, Nelson B, Karnezis A N, Damsky W E, Jr., You M J, DePinho R A, McMahon M, Bosenberg M. Braf (V600E) cooperates with Pten loss to induce metastatic melanoma. Nat Genet. 2009; 41(5): 544-52. Epub 2009/03/14. doi: 10.1038/ng.356. PubMed PMID: 19282848; PMCID: PMC2705918.
65. Chen Y, Chalouni C, Tan C, Clark R, Venook R, Ohri R, Raab H, Firestein R, Mallet W, Polakis P. The melanosomal protein PMEL17 as a target for antibody drug conjugate therapy in melanoma. J Biol Chem. 2012; 287(29): 24082-91. Epub 2012/05/23. doi: 10.1074/jbc.M112.361485. PubMed PMID: 22613716; PMCID: PMC3397835.
66. Dextras C, Dashnyam M, Griner L A M, Sundaresan J, Chim B, Yu Z, Vodnala S, Lee C R, Hu X, Southall N, Marugan J J, Jadhav A, Restifo N P, Acquavella N, Ferrer M, Singh A. Identification of Small Molecule Enhancers of Immunotherapy for Melanoma. Sci Rep. 2020; 10(1): 5688. Epub 2020/04/02. doi: 10.1038/s41598-020-62369-1. PubMed PMID: 32231230; PMCID: PMC7105471.
67. Peck B, Schulze A. Lipid Metabolism at the Nexus of Diet and Tumor Microenvironment. Trends Cancer. 2019; 5(11): 693-703. Epub 2019/11/19. doi: 10.1016/j.trecan.2019.09.007. PubMed PMID: 31735288.
68. Allen S, Osorio O, Liu Y-G, Scott E. Facile assembly and loading of theranostic polymersomes via multi-impingement flash nanoprecipitation. Journal of Controlled Release. 2017; 262: 91-103. doi: https://doi.org/10.1016/j.jconre1.2017.07.026.
69. Scott E A, Nichols M D, Kuntz-Willits R, Elbert D L. Modular scaffolds assembled around living cells using poly(ethylene glycol) microspheres with macroporation via a non-cytotoxic porogen. Acta Biomater. 2010; 6(1): 29-38. doi: 10.1016/j.actbio.2009.07.009. PubMed PMID: 19607945; PMCID: PMC2787810.
70. Belkina A C, Ciccolella C O, Anno R, Halpert R, Spidlen J, Snyder-Cappione J E. Automated optimized parameters for T-distributed stochastic neighbor embedding improve visualization and analysis of large datasets. Nat Commun. 2019; 10(1): 5415. Epub 2019/11/30. doi: 10.1038/s41467-019-13055-y. PubMed PMID: 31780669; PMCID: PMC6882880.

1. A multi-nanoparticle formulation comprising:
(1) an endocytosis inhibitory nanoparticle comprising:
(a) a nanostructure comprising a poly(ethylene glycol)-block-poly(propylene sulfide) (PEG-b-PPS) copolymer; and
(b) a endocytosis inhibitor loaded in the nanostructure; and
(2) an effector nanoparticle (E-NP).

2. The formulation of claim 1, wherein the endocytosis inhibitory nanoparticle is a macropinocytosis inhibitory nanoparticle (MiNP).

3. The formulation of claim 1, wherein the effector nanoparticle comprises a poly(ethylene glycol)-block-poly(propylene sulfide) (PEG-b-PPS) copolymer.

4. The formulation of claim 3, wherein the effector nanoparticle further comprises a targeting moiety incorporated into the surface of the nanoparticle, optionally the targeting moiety comprising:
(a) a cell-receptor-binding molecule;
(b) a polyethylene glycol (PEG) linker; and
(c) an anchor.

5. The formulation of claim 4, wherein (a) the cell-receptor-binding molecule is folate, (b) the anchor is a palmitoleic acid lipid anchor or both (a) and (b).

6. The formulation of claim 1, wherein the effector nanoparticle further comprises a therapeutic payload.

7. The formulation of claim 1, wherein the nanostructure is a micelle or a bicontinuous nanosphere.

8. A method for enhancing circulation time and/or cell-targeting efficacy of an effector nanoparticle in a subject, the method comprising administering the formulation of claim 1.

9. The method of claim 8, wherein the administering is performed subcutaneously or intravenously.

10. The method of claim 9, wherein uptake of the effector nanoparticle by the mononuclear phagocyte system (MPS) is inhibited.

11. The method of claim 8, wherein the method does not inhibit receptor-mediated endocytosis.

12. The method of claim 8, wherein the cell is a tumor cell.

13. The method of claim 8, wherein the administering is performed subcutaneously and accumulation of the effector nanoparticle in serum of the subject is increased.

14. A method for enhancing circulation time and/or cell-targeting efficacy of an effector nanoparticle in a subject, the method comprising the steps of:
(1) administering a endocytosis inhibitory nanoparticle comprising
(a) nanostructure comprising a poly(ethylene glycol)-block-poly(propylene sulfide) (PEG-b-PPS) copolymer; and
(b) a endocytosis inhibitor loaded in the nanostructure; and
(2) administering an effector nanoparticle.

15. The method of claim 14, wherein (a) the endocytosis inhibitor is a macropinocytosis inhibitor, (b) the effector nanoparticle comprises a poly(ethylene glycol)-block-poly(propylene sulfide) (PEG-b-PPS) copolymer, or (c) both (a) and (b).

16. The method of claim 14, wherein the nanostructure is a micelle or a bicontinuous nanosphere.

17. The method of claim 14, wherein the effector nanoparticle further comprises a targeting moiety incorporated into the surface of the nanoparticle, optionally the targeting moiety comprising:
(a) a cell-receptor-binding molecule;
(b) a polyethylene glycol (PEG) linker; and
(c) an anchor.

18. The method of claim 14, wherein the effector nanoparticle further comprises a therapeutic payload.

19. The method of claim 14, wherein the endocytosis inhibitory nanoparticle is administered prior to the effector nanoparticle.

20. The method of claim 14, wherein the endocytosis inhibitory nanoparticle is administered simultaneously with the effector nanoparticle.

21. The method of claim 14, wherein the administering is performed subcutaneously or intravenously.

22. The method of claim 14, wherein non-specific uptake of the effector nanoparticle by endocytosis is inhibited.

23. The method of claim 14, wherein the method does not inhibit receptor-mediated endocytosis.

24. The method of claim 17, wherein the cell is a tumor cell.

25. The method of claim 14, wherein the administering is performed subcutaneously and accumulation of the effector nanoparticle in serum of the subject is increased.
